(19) United States
(12) Patent Application Publication
(10) Pub. No.: US 2026/0035410 A1
(43) Pub. Date: Feb. 5, 2026

(54) THERAPEUTIC PEPTIDES AND METHODS OF PEPTIDE DESIGN

(71) Applicants: Wisconsin Alumni Research Foundation, Madison (WI); University of Illinois - Chicago, Chicago, IL (US)

(72) Inventors: Seungpyo Hong, Madison, WI (US); Petr Král, Evanston, IL (US); Piper Rawding, Madison, WI (US); Francesco Coppola, San Cesario di Lecce (IT)

(73) Assignees: Wisconsin Alumni Research Foundation, Madison, WI (US); University of Illinois - Chicago, Chicago, IL (US)

(21) Appl. No.: 19/288,573

(22) Filed: Aug. 1, 2025

Related U.S. Application Data
(60) Provisional application No. 63/678,427, filed on Aug. 1, 2024.

(51) Int. Cl. C07K 7/08; A61K 9/51; A61K 38/00; A61P 37/02; C07K 7/64; C12N 15/10

(52) U.S. Cl. CPC C07K 7/08; A61K 9/5169; A61P 37/02; C07K 7/64; C12N 15/1037; A61K 38/00

(57) ABSTRACT

Described herein are TIGIT binding peptides, nanoparticle systems such as dendrimer systems including the TIGIT binding peptides, pharmaceutical formulations, and methods of use. Also described are methods of identifying peptides that bind a target receptor, the method including phage display, chemical synthesis, determination of binding affinities, and computational modeling. Advantageously, adaptive evolution modeling can be used to optimize the peptides identified in the methods for improved binding affinities.

### CROSS-REFERENCE TO RELATED APPLICATIONS

This application claims priority to U.S. Provisional Application 63/678,427 filed on Aug. 1, 2024, which is incorporated herein by reference in its entirety.

### STATEMENT REGARDING FEDERALLY SPONSORED RESEARCH & DEVELOPMENT

This invention was made with government support under 2211932 awarded by the National Science Foundation. The government has certain rights in the invention.

### SEQUENCE LISTING

The Instant Application contains a Sequence Listing which has been submitted electronically in XML format and is hereby incorporated by reference in its entirety. Said XML copy, created on Jul. 30, 2025, is named “SEQ_LIST--107668333-P240330US02.xml” and is 15,411 bytes in size. The Sequence Listing does not go beyond the disclosure in the application as filed.

### FIELD OF THE DISCLOSURE

The present disclosure is related to methods of identifying therapeutic peptides, therapeutic peptides, nanoparticle constructs comprising the therapeutic peptides, and methods of use thereof.

### BACKGROUND

The utilization of peptides for immunotherapy has attracted a great deal of interest due to their ability to mimic protein functionalities while maintaining a high degree of modularity and synthetic reproducibility in their molecular design. In addition, the clinical utility of a multitude of artificial bioactive peptides with enhanced anti-tumor therapeutic outcomes has been demonstrated. However, the clinical translation of otherwise promising peptide-based immunotherapeutics has been hindered due to their significantly weaker binding to target proteins and short half-life in plasma circulation in comparison to their monoclonal antibody counterparts. As described in U.S. Pat. No. 11,564,995, these issues can be addressed by conjugation of therapeutic peptides to nanoparticles, which enhances their cell binding/uptake and prolongs their circulation. Dendrimer-peptide conjugates (DPCs), for example, can facilitate simultaneous binding between its multiple peptides and cell receptors, resulting in a significantly enhanced binding avidity through the multivalent binding effect. DPCs can advantageously have case of manufacture (resulting in minimal batch-to-batch variations), reduced immunogenicity, ability to control sequence and structure, and lower cost.

Peptide development, however, can be a long and arduous process resulting in large libraries of peptides that need to be synthesized, screened experimentally, and modified to improve pharmacological properties. Described herein are novel methods combining experimental and modeling approaches for the reliable and rapid identification and development of therapeutic peptides, the therapeutic peptides, and nanoparticle compositions comprising the therapeutic peptides.

### BRIEF SUMMARY

In one aspect, a T cell immunoreceptor with Ig and ITIM domains (TIGIT)-binding peptide comprises the amino acid sequence of any of SEQ ID NOs. 1-14. In another aspect, a TIGIT-binding peptide comprises the amino acid sequence of any of SEQ ID NOs. 1-14 with 1-3 amino acid substitutions, wherein the TIGIT-binding peptide binds mouse TIGIT (SEQ ID NO: 15) or human TIGIT (SEQ ID NO: 16) with a dissociation constant of 10⁻³ M or less.

In another aspect, a nanoparticle system comprises a multivalent nanoparticle core comprising a plurality of TIGIT-binding peptides conjugated thereto. The plurality of TIGIT-binding peptides may be the same or different.

In a further aspect, included are pharmaceutical compositions comprising the foregoing nanoparticle system and a pharmaceutically acceptable excipient.

In another aspect, an immunotherapy method comprises administering the pharmaceutical composition to a subject in need of immunotherapy.

In a still further aspect, a method of making a nanoparticle system comprises contacting multivalent nanoparticle cores comprising multiple reactive end groups with a composition comprising one or more TIGIT-binding peptides as described above under conditions sufficient to conjugate a plurality of the peptides to the multivalent nanoparticle cores and provide the nanoparticle system.

In another aspect, a method of identifying a therapeutic peptide which binds a target receptor comprises
a) performing phage display with a phage display library to identify candidate target receptor binding peptides,
b) sequencing DNA encoding the candidate target receptor binding peptides,
c) synthesizing the candidate target receptor binding peptides and determining the binding affinity of the synthesized candidate target receptor binding peptides to the target receptor,
d) based on the binding affinities of c), performing computational modeling to determine conserved peptide-receptor binding components, and
e) performing adaptive-evolution modeling and identifying one or more mutated target receptor binding peptides with optimized peptide-receptor binding components,
f) synthesizing the one or more mutated target receptor binding peptides and determining the binding affinity of the synthesized mutant target receptor binding peptides to the target receptor, and
g) identifying the therapeutic peptide as a peptide from f) having a dissociation constant of at least 10⁻³ M or lower for the target receptor.

### BRIEF DESCRIPTION OF THE DRAWINGS

FIGS. 1A and B show TIGIT-binding peptides and sequence alignment. 1A shows the structure of identified TIGIT-binding peptide analogs from liquid biopanning experiments. 1B shows the sequence alignment and consensus analysis of the identified peptide analogs (TBP-23 (SEQ ID NO: 8); TBP-22 (SEQ ID NO: 7), TBP-8 (SEQ ID NO: 4), TBP-2 (SEQ ID NO: 2), TBP-7 (SEQ ID NO: 3), TBP-13 (SEQ ID NO: 5)).

FIGS. 2A and B show relative binding affinity analyzed using biolayer interferometry (BLI). 2A shows dissociation constants and 2B shows binding curves from BLI experiments. The observed dissociation constant (Kp) values (M) are as follows: 2-4.61E-5±2.09E-5; 7-3.45E-4±2.00 E-4; 8-1.14E-4±1.04E-4; 13-1.2E-4±1.08E-4; 22-9.09E-4±5.24E-4; and 23-2.03E-4±1.09E-4.

FIGS. 3A-C show the results of simulations of binding interactions of the peptides. 3A shows the results of a simulation showing interaction energies of each analog interacting with TIGIT receptors over 500 nanoseconds. 3B shows the crystal structure of TIGIT-polio virus receptor (PVR, CD155) complex that shows the critical region of interaction. It has been observed that the TIGIT-PVR receptor-ligand pair interacts through conserved motifs that form a specific lock and key interaction. 3C shows an analysis of analogs 22 and 23, which were found to most specifically interact with TIGIT, looking at the residue location and number of interactions observed between the analogs and receptor.

FIGS. 4A-C show optimization of the identified conservative binding regions. 4A shows a simulation showing interaction energies of each analog interacting with TIGIT receptors over 500 nanoseconds, demonstrating the mutant peptide's significantly enhanced binding affinity to the TIGIT receptor relative to the other analogs. 4B is a model showing the mutant peptide (licorice) interacting with the TIGIT receptor and isolated mutant peptide's structure. 4C shows dissociation constants from BLI, experimentally confirming the mutant peptide's enhanced affinity.

FIGS. 5A-C show in vitro characterization of TIGIT-binding peptides and DPCs' effect on NK cell-mediated cytotoxicity. 5A shows the change in cell count relative to null (untreated) controls. 5B shows the percent Calcein release from K7M2. 5C shows representative images from the experiment, illustrating the potent inhibitory effects of the mutant dendrimer-protein conjugate (DPC). Groups were compared using Prism v10 software (GraphPad) using Brown-Forsythe and Welch one-way ANOVA tests. Differences were considered statistically significant when p≤0.05.

The above-described and other features will be appreciated and understood by those skilled in the art from the following detailed description, drawings, and appended claims.

### DETAILED DESCRIPTION

Described herein is a method for identifying therapeutic peptides which combines traditional synthetic and analytical chemistry methods to identify high-affinity peptide sequences and predictive computational techniques to optimize their binding to target receptors. Phage display is a biological technique based on the ability of bacteriophages to express foreign proteins on their surface. In this cyclic process, first, a display library is incubated with an immobilized protein target. Then, the loose phages are cluted, and remaining antigen-specific phages are enriched by amplification in host cells. This process is cyclically repeated until phage clones are identified that specifically bind to the target. Peptides binding with a relatively high affinity to antigen proteins could be obtained by sequencing the remaining phage clones. These candidates can then be synthesized using solid-phase peptide synthesis (SPPS) and tested experimentally (biolayer interferometry, BLI, or surface plasmon resonance, SPR, analysis) to check their binding affinity to antigens. Next, computational modeling (e.g., Nanoscale Molecular Dynamics, NAMD, with Chemistry at Harvard Macromolecular Mechanics, CHARMM, force field) may be used to identify favorable conserved regions in peptide-antigen binding components. Then, adaptive evolution modeling is used to design and optimize mutant peptide analogs capable of blocking antigen proteins. Finally, these mutant peptides may also be prepared and experimentally tested.

Specifically, several highly selective sequences were identified that bind the mouse ortholog of TIGIT (T cell immunoreceptor with Ig and ITIM domains). Given the similarity between mouse and human TIGIT, it is predicted the peptides will also bind human TIGIT, or can be modified with 1-3 amino acid substitutions to bind human TIGIT. TIGIT is an inhibitory receptor expressed on lymphocytes that has been identified as a potential therapeutic target to enhance anti-tumor immune responses. TIGIT is expressed by activated CD8⁺ T and CD4⁺ T cells, natural killer (NK) cells, regulatory T cells (Tregs), and follicular T helper cells in humans. TIGIT blockade is a promising immunotherapy for cancer, particularly when combined with PD-1 blockade.

### Method of Identifying Therapeutic Peptides

In an aspect, described herein is a method of identifying a therapeutic peptide which binds a target receptor, comprising
a) performing phage display with a phage display library to identify candidate target receptor binding peptides,
b) sequencing the DNA encoding the candidate target receptor binding peptides,
c) synthesizing the candidate target receptor binding peptides and determining the binding affinity of the synthesized candidate target receptor binding peptides to the target receptor,
d) based on the binding affinities of c), performing computational modeling to determine conserved peptide-receptor binding components,
e) using the conserved peptide-receptor binding components, performing adaptive-evolution modeling and identifying one or more mutated target receptor binding peptides with optimized peptide-receptor binding components,
f) synthesizing the one or more mutated target receptor binding peptides and determining the binding affinity of the synthesized mutant target receptor binding peptides to the target receptor, and
g) identifying the therapeutic peptide as a peptide from f) having a having a dissociation constant of 10⁻³ M, 10⁻⁴ M, 10⁻⁵ M, 10⁻⁶ M or 10⁻⁷ M or lower for the target receptor.

Phage display is a selection technique in which a library of peptides is expressed on the outside of a phage. The DNA encoding each peptide variant is inside the phage creating a link between each variant protein sequence and the encoding DNA. Using a selection process called panning or biopanning, which allows rapid partitioning based on binding affinity to a given target molecule, the phages that bind a target molecule (e.g., a target receptor) are selected. An exemplary selection method comprises:
i) providing the phage display library expressing a plurality of test peptides,
ii) incubating the phage display library with the target receptor, wherein the target receptor is optionally immobilized,
iii) selecting phage bound to the target receptor,
iv) amplifying the selected phage of iii), and
v) repeating steps ii) to iv) one or more times to provide the candidate target receptor binding peptides.

Exemplary target receptors for the selection method described herein are immune checkpoint axes of interest including TIGIT, VISTA/VSIG-3, TIM-3/Galectin9, LAG-3/LSECtin/FGL1, GITR/GITRL, and CTLA-4/CD80.

A “phage display library” means a collection of phages that have been genetically engineered to express a set of test peptides on their outer surface. In embodiments, DNA sequences encoding the test peptides are inserted in frame into a gene encoding a phage capsule protein. In other embodiments, the putative targeting peptide sequences are in part random mixtures of all twenty amino acids and in part non-random. In certain embodiments the putative targeting peptides of the phage display library exhibit one or more cysteine residues at fixed locations within the targeting peptide sequence. While there is no particular limit on the length of the test peptides, lengths of 8-20, preferably 10-15 amino acids may be employed. A typical phage display library includes 10⁸-10⁹ peptides and may include about 100 copies of each variant peptide.

A “receptor” for a peptide includes but is not limited to any molecule or complex of molecules that binds to a peptide. Non-limiting examples of receptors include peptides, proteins, glycoproteins, lipoproteins, epitopes, lipids, carbohydrates, multi-molecular structures, a specific conformation of one or more molecules and a morphoanatomic entity.

Steps ii) to iv) are referred to as a “round” or “cycle” of biopanning, and generally 3-4 rounds or cycles are performed before characterizing individual clones. In step ii), the target receptor may be immobilized on a surface or bead to facilitate selection of phage variants which bind the target receptor. The selected (i.e., bound phage) are separated from the unbound phage and then amplified (e.g., in E. coli) prior to performing another round of selection. In general, 3-4 rounds of selection provides enrichment of phage which bind the target receptor.

Once the candidate target receptor binding peptides are identified in a), in step b) the DNA encoding the candidate target receptor binding peptides is sequenced, for example using Sanger sequencing.

In step c) once the sequence of the candidate target receptor binding peptides is identified, the candidate target receptor binding peptides are synthesized (e.g., using solid phase synthesis), and the binding affinity of the synthesized candidate target receptor binding peptides to the target receptor is determined. Determining the binding affinity to the target receptor can comprise biolayer interferometry (BLI), or surface plasmon resonance (SPR). Step c) optionally further comprises performing docking simulations of the candidate target receptor binding peptides on the structure of the target receptor.

Based on the binding affinities determined in step c), in step d) computational modeling (e.g., molecular dynamics simulation) is performed to determine conserved peptide-receptor binding components. Computational modeling can comprise Visual Molecular Dynamics and scalable molecular dynamics with an NAMD package using CHARMM general and protein forcefields. Conserved peptide-receptor binding components can include common interaction sited among the candidate target receptor binding peptides, including distinct and shared binding pockets.

In step e), adaptive-evolution modeling is used to identify mutated target receptor binding peptides with optimized peptide-receptor binding components. For example, iterative Monte Carlo (MC) simulations can be used to explore diverse arrangements and optimize sequences. Short molecular dynamics simulations of the mutated peptides interacting with the TIGIT receptor can be conducted, followed by MC sampling using a Metropolis criterion to accept or reject mutations based on changes in the binding interaction free energy. The mutation/selection cycle can be iteratively repeated until a mutant peptide with a satisfactory predicted binding affinity is identified. In certain embodiments, a mutant peptide with dissociation constant for the target receptor in the nM range may be preferred, although peptides with mM dissociation constants can be employed.

In step f), the one or more mutated target receptor binding peptides are synthesized and the binding affinity of the synthesized mutant target receptor binding peptides to the target receptor are determined as described above.

Finally, in step g), a peptide is identified as the therapeutic peptide when the peptide from step f) has a dissociation constant of at least 10⁻³ M, preferably at least 10⁻⁴ M, more preferably at least 10⁻⁶ M, and most preferably at least 10⁻⁷ M for the target receptor.

In an aspect, the method can further comprise h) synthesizing the therapeutic peptide and conjugating the therapeutic peptide to a multivalent nanoparticle core.

### TIGIT Binding Peptides and Nanoparticle Systems

In an aspect, described herein are peptides that bind TIGIT. Exemplary peptides are listed in Table 1, wherein SEQ ID NOs. 1-8 are peptides identified by phage display and tested experimentally, and SEQ ID NOs. 9 and 10 are mutant peptides identified by computational modeling and adaptive evolution modeling. SEQ ID NOs. 11-14 were also identified by computational modeling and adaptive evolution modeling.

TABLE 1
TIGIT binding peptides

| | | SEQ |
|---|---|---|
| | | ID |
| Analog | Sequence | NO: |
| 2 | QVACHWMCTDQT | 1 |
| 3 | NDAVIKNLLSSN | 2 |
| 7 | NGSQSDIYRIQT | 3 |
| 8 | ITLGYDARAPNA | 4 |
| 13 | VDSYSPPTTSHL | 5 |
| 21 | HDLGQYIIYGQM | 6 |
| 22 | DEGHVTSKHYNW | 7 |
| 23 | AFWKTDGFSRAN | 8 |
| M1 | YPWRWYYHVTFFCRW | 9 |
| M2 | TKGHVTSKHYNW | 10 |
| TIGIT-TIGIT interface (TT) | RTRWYRGVTMGEW | 11 |
| TIGIT-PVR interface (TP1) | QYFPENRVMERAWR | 12 |
| TIGIT-PVR interface (TP2) | QYFPMTRVMERAWR | 13 |
| TIGIT-PVR interface (TP3) | QYFPENRCMERAWR | 14 |

As used herein, an “amino acid residue” refers to any naturally occurring amino acid, any amino acid derivative or any amino acid mimic known in the art. In certain embodiments, the residues of the protein or peptide are sequential, without any non-amino acid interrupting the sequence of amino acid residues. In other embodiments, the sequence may comprise one or more non-amino acid moieties. In particular embodiments, the sequence of residues of the protein or peptide may be interrupted by one or more non-amino acid moieties. Accordingly, the term “protein or peptide” encompasses amino acid sequences comprising at least one of the 20 common amino acids found in naturally occurring proteins, or at least one modified or unusual amino acid.

In an aspect, included herein is a peptide comprising the amino acid sequence of any of SEQ ID NOs. 1-14. In another aspect, included herein is a peptide comprising the amino acid sequence of any of SEQ ID NOs. 1-14 with 1-5, preferably 1-4, and more preferably 1-3 amino acid substitutions, wherein the TIGIT-binding peptide binds mouse TIGIT (SEQ ID NO: 15, UNIPROT P86176) or human TIGIT (SEQ ID NO: 16, UNIPROT Q495A1) with a dissociation constant of 10⁻³ M or less.

In an aspect, the peptide is conjugated to a diagnostic or imaging agent.

Diagnostic agents are agents that enable the detection or imaging of a tissue or disease. Examples of diagnostic agents include, but are not limited to, radiolabels, fluorophores and dyes.

Imaging agent refers to a label that is attached to the peptide for imaging a tumor, organ, or tissue in a subject. Examples of imaging agents include, without limitation, radionuclides, fluorophores such as fluorescein, rhodamine, isothiocyanates (TRITC, FITC), Texas Red, Cy2, Cy3, Cy5, APC, and the AlexaFluor® (Invitrogen, Carlsbad, Calif.) range of fluorophores, antibodies, gadolinium, gold, nanomaterials, horseradish peroxidase, alkaline phosphatase, derivatives thereof, and mixtures thereof.

Radiolabel refers to a nuclide that exhibits radioactivity. A “nuclide” refers to a type of atom specified by its atomic number, atomic mass, and energy state, such as carbon 14 (¹⁴C). “Radioactivity” refers to the radiation, including alpha particles, beta particles, nucleons, electrons, positrons, neutrinos, and gamma rays, emitted by a radioactive substance.

In an aspect, the peptide is circularized. Methods of preparing cyclic peptides are known in the art and include chemical methods such as linking the N- and C-terminus, using an amino acid sidechain to form a cyclic amide bond, using disulfide chemistry, using click chemistry, and the like.

In an embodiment, a nanoparticle system comprises a multivalent nanoparticle core comprising a plurality of therapeutic peptides conjugated thereto. The plurality of therapeutic peptides can include multiples of the same therapeutic peptide, or different therapeutic peptides conjugated to the same nanoparticle core. In specific embodiments, the multivalent nanoparticle core comprises a hyperbranched polymer, a dendrimer, a dendron, a hybrid nanoparticle, or a micelle. The multivalent nanoparticle cores can have diameters of 3 to 150 nm, for example.

As used herein, hyperbranched polymers are multivalent particles that are polydisperse and irregular in terms of their branching and structure. Dendrimers, in contrast, have a very regular, radially symmetric generation structure. Dendrimers are monodisperse globular polymers which, by comparison with hyperbranched polymers, are typically prepared in multistep syntheses. The dendrimer structure is characterized by the polyfunctional core which represents the center of symmetry, various well-defined radially symmetric layers of a repeating unit (generation) and the terminal groups.

Hyperbranched polymers include polyesters, polyesteramides, polyethers, polyamides, polyethylencimines, polyglycerols, polyglycolides, polylactides, polylactide-co-glycolides, polytartrates, and polysaccharides. Hyperbranched polyesters include Boltorn® from Perstorp AB, hyperbranched polyesteramides include Hybrane® from DSM BV Niederlande, polyglycerols are produced by Hyperpolymers GmbH, and hyperbranched polyethyleneimines include Polyimin® from BASF AG.

Hyperbranched polymers also include polycaprolactones and copolymers such as poly(D,L-lactide-co-glycolides) and the polyester compounds produced by Degussa AG from the Dynapo® S and Dynacoll® product families.

Preparation of hyperbranched polymers, e.g., hyperbranched polyglycerols, is well known in the art. For example, controlled anionic ring-opening multibranching polymerization of glycidol is performed to form hyperbranched polyglycerols. Hyperbranched polyglycerols are then reacted with succinic anhydride in pyridine to provide carboxylic acid terminal groups via an ester linkage. Once the functional group content on hyperbranched polyglycerols is verified, the hydroxyl can be further functionalized by the following scheme: hyperbranched polyglycerols-OH+N-(p-maleimidophenyl) isocyanate (PMPI, 10-fold molar excess) in DMSO or DMF at pH 8.5 to obtain hyperbranched polyglycerols-maleimide. Hyperbranched polyglycerols thus possess both carboxyl and maleimide functional groups that can react with corresponding cross-linkers and chemical groups or can be further derivatized to suit specific functional groups available.

Amphiphilic hyperbranched polymers can form micelle-like structures. The hyperbranched polymer can be an “imperfect” molecule, in that it may include linear sections, and may feature random or unsymmetrical branching. Hyperbranched polymers can be selectively modified to achieve multiple functionalities on the surface and linked to functional components such as carbon chains to install hydrophobicity, and primary amine groups for hydrophilicity and activation for subsequent modifications.

The advantages of hyperbranched polymers include smaller unit sizes (typically <60 nm in diameter) and relatively simple procedures for synthesis. Potential disadvantages include broad size distributions and potential difficulties controlling surface modification for specific functionalities.

The term “dendrimer” as used herein includes, but is not limited to, a molecular architecture with an interior core, interior layers (or “generations”) of repeating units regularly attached to and extending from this initiator core, each layer having one or more branching points, and an exterior surface of terminal groups attached to the outermost generation. Dendrimers have regular dendrimeric or “starburst” molecular structures. Nanoparticle dendrimers generally have diameters of 3 to 10 nm, for example.

Each successive dendrimer generation can be covalently bound to the previous generation. The number of reactive groups of the core structure determines n-directionality and defines the number of structures that can be attached to form the next generation.

The number of branches in a dendritic structure is dependent on the branching valency of the monomeric building blocks, including the core. For example, if the core is a primary amine, the amine nitrogen would then be divalent, resulting in a 1-2 branching motif.

Exemplary dendrimers are alkylated dendrimers such as poly(amido-amine) (PAMAM), poly(ethyleneimine) (PEI), polypropyleneimine (PPI), diaminobutane amine polypropylenimine tetramine (DAB-Am 4), polypropylamine (POPAM), polylysine, polyester, iptycene, aliphatic poly(ether), aromatic polyether dendrimers, or a combination comprising one or more of the foregoing.

The dendrimers can have carboxylic, amine and hydroxyl terminations and can be of any generation including, but not limited to, generation 1 dendrimers (G1), generation 2 dendrimers (G2), generation 3 dendrimers (G3), generation 4 dendrimers (G4), generation 5 dendrimers (G5), generation 6 dendrimers (G6), generation 7 dendrimers (G7), generation 8 dendrimers (G8), generation 9 dendrimers (G9), or generation 10 dendrimers (G10).

The PAMAM dendrimers contain internal amide bonds which may enhance their biodegradability, thus improving tolerance in terms of human therapeutic applications. The surface includes polar, highly reactive primary amine groups. The surfaces of the amino-functional PAMAM dendrimers are cationic and can be derivatized, either through ionic interactions with negatively charged molecules, or using many well-known reagents for covalent functionalization of primary amines.

When PAMAM dendrimers are employed, generations from 0 to 7 PAMAM dendrimers are typically used. For example, hybrid nanoparticles can be formed from generation 0 PAMAM dendrimers (G0); generation 1 (G1) PAMAM dendrimers; generation 2 (G2) PAMAM dendrimers; generation 3 (G3) PAMAM dendrimers; generation 4 (G4) PAMAM dendrimers; generation 5 (G5) PAMAM dendrimers; generation 6 (G6) PAMAM dendrimers; or generation 7 (G7) PAMAM dendrimers. PAMAM is commercially available from multiple sources, including Sigma-Aldrich (Cat. No. 597309).

Diaminobutane amine polypropylenimine tetramine (DAB Am 4) is a polymer with a 1,4-diaminobutane core (4-carbon core) with 4 surface primary amino groups. When hybrid nanoparticles are formed from DAB-AM 4 dendrimers, generations from 0 to 7 DAB-AM 4 dendrimers are typically used. For example, hybrid nanoparticles can be formed from generation 0 DAB-AM 4 dendrimers (G0); generation 1 (G1) DAB-AM 4 dendrimers; generation 2 (G2) DAB-AM 4 dendrimers; generation 3 (G3) DAB-AM 4 dendrimers; generation 4 (G4) DAB-AM 4 dendrimers; generation 5 (G5) DAB-AM 4 dendrimers; generation 6 (G6) DAB-AM 4 dendrimers; or generation 7 (G7) DAB-AM 4 dendrimers. DAB-Am 4 is commercially available from multiple sources, including Sigma-Aldrich (Cat. No. 460699).

The multivalent nanoparticles may be formed of one or more different dendrimers. Each dendrimer of the dendrimer complex may be of similar or different chemical nature than the other dendrimers (e.g., the first dendrimer can be a PAMAM dendrimer, while the second dendrimer can in be a POPAM dendrimer).

In an aspect, a portion of the surface groups of the dendrimer can be chemically modified, e.g., acetylated or methoxylated, to reduce the number of available peptide binding sites. This can reduce crowding of peptides on the surface and improve the function of the complexes. In an aspect, 40% to 90%, specifically 60% to 90% of the end groups of the dendrimer are modified.

Dendrons are monodisperse, wedge-shaped dendrimer sections with multiple terminal groups and a single reactive function at the focal point. Dendrons can be grafted to a surface, another dendron, or a macromolecule, for example. Bis-MPA (bis-dimethylolpropionic acid) dendrons are available from Sigma-Aldrich.

As used herein, a “micelle” refers to an aggregate of amphiphilic molecules in an aqueous medium, having an interior core and an exterior surface, wherein the amphiphilic molecules are predominantly oriented with their hydrophobic portions forming the core and hydrophilic portions forming the exterior surface. Various monoclonal antibodies, peptides, proteins, and small molecules can covalently bind to the hydrophilic head group of micelles, covering the nanoparticle with plurality of conjugated ICIs for stronger binding kinetics. Micelles are typically in a dynamic equilibrium with the amphiphilic molecules or ions from which they are formed existing in solution in a non-aggregated form. Many amphiphilic compounds, including in particular detergents, surfactants, amphiphilic polymers, lipopolymers (such as PEG-lipids), bile salts, single-chain phospholipids and other single-chain amphiphiles, and amphipathic pharmaceutical compounds are known to spontaneously form micelles in aqueous media above certain concentration, known as critical micellization concentration, or CMC. The amphipathic, e.g., lipid, components of a micelle do not form bilayer phases, nonbilayer mesophases, isotropic liquid phases or solid amorphous or crystalline phases. The concept of a micelle, as well as the methods and conditions for their formation, are well known to skilled in the art. Micelles can co-exist in solution with lipidic particles.

Exemplary micelles include those described in U.S. Pat. No. 9,212,258, incorporated by reference for its disclosure of micelles comprising amphiphilic dendron-coils. Each amphiphilic dendron-coil comprises a non-peptidyl, hydrophobic core-forming block, a polyester dendron and a poly(ethylene) glycol (PEG) moiety. The micelles comprising amphiphilic dendron-coils are also referred to as “multivalent dendron conjugates” and “dendron-based nanomicelles (DNMs)”.

The hydrophobic core-forming block of the micelles is non-peptidyl, that is, the hydrophobic core-forming block is not a peptide. In some embodiments, a micelle comprises a single type of amphilphilic dendron-coil (i.e., the amphiphilic dendron-coils in the micelle all have the same three components.) In some embodiments, a micelle comprises more than one type of amphiphilic dendron-coil (i.e., the amphiphilic dendon-coils in the micelle vary in their three components.)

In some embodiments, the non-peptidyl, hydrophobic core-forming block of the amphiphilic dendron-coil comprises polycaprolactone (PCL), poly(lactic acid) (PLA), poly(glycolic acid) (PGA) or poly(lactic-co-glycolic acid) (PLGA). In some embodiments, the non-peptidyl, hydrophobic core-forming block is PCL. In some embodiments, the PCL is poly(ε-caprolactone). In some embodiments, the non-peptidyl, hydrophobic core-forming block is PLA. In some embodiments, the non-peptidyl, hydrophobic core-forming block is PGA. In some embodiments, the non-peptidyl, hydrophobic core-forming block is PLGA. The non-peptidyl, hydrophobic core-forming block has a molecular weight including, but not limited to, a molecular weight of about 0.5 kDa to about 20 kDa. In some embodiments, the non-peptidyl, hydrophobic core-forming block is poly(ε-caprolactone) with a molecular weight of about 3.5 kDa. In some embodiments, the non-peptidyl, hydrophobic core-forming block is poly(ε-caprolactone) has a molecular weight of 14 kDa.

In some embodiments, the polyester dendron of the amphiphilic dendron-coil includes, but is not limited to, a generation 3 to generation 5, that is, a generation 3 (G3), a generation 4 (G4) or a generation 5 (G5), polyester dendron with either an acetylene or carboxylate core. In some embodiments, the polyester dendron is a G3 dendron. In some embodiments, the polyester dendron is a G5 dendron. In some embodiments, the polyester dendron has an acetylene core. In some embodiments, the polyester dendron is generation 3 polyester-8-hydroxyl-1-acetylene bis-MPA dendron. In some embodiments, the polyester dendron has a carboxylate core.

In some embodiments, the PEG moiety of the amphiphilic dendron-coil is a methoxy PEG (mPEG) moiety, amine-terminated PEG (PEG-NH2) moiety, acetylated PEG (PEG-Ac) moiety, carboxylated PEG (PEG-COOH) moiety, thiol-terminated PEG (PEG-SH) moiety, N-hydroxysuccinimide-PEG (PEG-NHS) moiety, NH2-PEG-NH2 moiety or NH2-PEG-COOH moiety. In some embodiments, the PEG moiety has a molecular weight including, but not limited to, a molecular weight from about 0.2 kDa to about 5 kDa. In some embodiments, the PEG moiety is an mPEG moiety. In some embodiments, the PEG moiety is an mPEG moiety with a molecular weight of about 2 kDa. In some embodiments, the PEG moiety is an mPEG moiety with a molecular weight of about 5 kDa.

In an embodiment, a polyester dendron is covalently modified with the linear hydrophobic polymer to help to facilitate chain entanglement and intramolecular interactions which aid in the self-assembly of core-shell type micelles and enable hydrophobic drug molecules to be loaded within the micelles. The PEG moieties form a hydrophilic corona with non-fouling properties and afford increased circulation half-life when the micelles are administered in vivo.

Biologically important properties such as biodegradability, circulation half-life, targetability, pharmacokinetics and drug release can be controlled by varying the three components (also referred to as the three polymer blocks) of the amphiphilic dendron-coils. Moreover, the copolymer structure is flexible and can be easily manipulated by varying the molecular weights of each component to fine-tune the hydrophilic-lipophilic balances (HLBs). For example, various embodiments employ PCL, polyester dendron, and PEG with molecular weights ranging 0.5-20 kDa, G3-G5 (approximately 0.9-3.5 kDa), and 0.2-5 kDa, respectively. The HLBs (20 M_{H}/(M_{H}+M_{L}), where M_{H} is the mass of the hydrophilic block and M_{L} is the mass of the lipophilic block) therefore widely vary from 2.22 to 19.94.

When a dendron is co-polymerized with the hydrophobic linear polymer such as polycaprolactone (PCL), poly(lactic acid) (PLA), poly(glycolic acid) (PGA), and poly(lactic-co-glycolic acid) (PLGA) in the generation of the amphiphilic dendron-coils, the cone-shaped, amphiphilic dendron-coils in turn possess advantageous structural attributes because they form self-assembled micelles, which are thermodynamically favorable and have highly packed PEG surface layers for increased blood circulation time. The thermodynamic stability in forming micelles, along with the unique architecture that is easily tunable.

The nanocarrier systems include hybrids of hyperbranched polymers and other biocompatible nanoparticles. For example, such hybrid nanoparticles include dendrimer-liposome, dendrimer-PEG-PLA, dendrimer-exosome hybrids that combine unique advantages of dendrimers (2-10 nm in diameter) and larger nanoparticles (50-200 nm).

Exemplary hybrid nanoparticles (also referred to as nanohybrids) include those described in U.S. Pat. No. 9,168,225, incorporated herein by reference for its disclosure of hybrid nanoparticles. In this embodiment, a hybrid nanoparticle is a particle in which a nanocore is surrounded or encapsulated in a matrix or shell. In other words, a smaller particle within a larger particle. In certain embodiments, the hybrid nanoparticles comprise a nanocore inside a liposome. In other embodiments, the nanocore is surrounded by a polymeric matrix or shell (e.g., a polymeric nanoparticle).

The nanocores are preferably from 1 nm to 50 nm in their greatest diameter. More preferably, the nanocores range from 1 to 40 nm in their greatest diameter, most preferably from 3 to 20 nm in their greatest diameter. The nanocores may be analyzed by dynamic light scattering and/or scanning electron microscopy to determine the size of the particles. A nanocore can have any shape and any morphology. Examples of nanocores include nanopowders, nanoclusters, nanocrystals, nanospheres, nanofibers, and nanotubes. Given its nanoscale size, the nanocore scaffold is readily excreted. Therefore, the nanocore scaffold employed need not be biodegradable, but in particular embodiments, the nanocore scaffold is biocompatible, i.e., not toxic to cells. Scaffolds are “biocompatible” if their addition to cells in vitro results in less than or equal to 30%, 20%, 10%, 5%, or 1% cell death and do not induce inflammation or other such unwanted adverse effects in vivo.

Exemplary polymeric scaffolds include, but are not limited to, a polyamide, a polysaccharide, a polyanhydride, poly-L-lysine, a polyacrylamide, a polymethacrylate, a polypeptide, a polyethylene oxide, a polyethyleneimine (PEI), or a dendrimer such as poly(amidoamine) (PAMAM) and PAMAM (ethylenediamine-EDA) dendrimers or modified versions thereof, e.g., hydroxylated, acetylated, or carboxylated versions of said polymers. Other exemplary polymeric backbones are described, e.g., in WO98/46270 (PCT/US98/07171) or WO98/47002 (PCT/US98/06963). The multivalent polymeric scaffold molecules can have a configuration selected from linear, branched, forked or star-like.

In some embodiments, at least a portion of the multivalent polymeric scaffold molecule may be hydrophobic. In some embodiments, at least a portion of the multivalent polymeric scaffold molecule may be hydrophilic. In another embodiment, a portion of the multivalent polymeric scaffold molecule may be hydrophobic, and a different portion of the molecule may be hydrophilic. In particular embodiments, the multivalent polymeric scaffold molecule is cationic. In other embodiments, the multivalent polymeric scaffold molecule is electronically neutral. In still other embodiments, the multivalent polymeric scaffold molecule is anionic. Those skilled in the art will recognize that various starting materials may be selected to obtain a multivalent polymeric scaffold molecule that exhibits the desired properties.

In one embodiment, the shell is a liposome composed of a phospholipid such as egg phosphatidylcholine, egg phosphatidylethanolamine, soy bean phosphatidylcholine, lecithin, sphingomyelin, synthetic phosphatidylcholine, lyso-phosphatidylcholine, phosphatidylglycerol, phosphatidic acid, phosphatidylethanolamine, or phosphatidylserine, wherein the phospholipid can be modified with a long-circulating agent or cryoprotectant. In another embodiment, the shell is polymeric nanoparticle composed of a polymer selected from the group of poly-(γ-L-glutamylglutamine), poly-(γ-L-aspartylglutamine), poly-L-lactic acid, poly-(lactic acid-co-glycolic acid), polyalkylcyanoacrylate, polyanhydrides, polyhydroxyacids, polypropylfumerate, polyamide, polyacetal, polyether, polyester, poly(orthoester), polycyanoacrylate, [N-(2-hydroxypropyl)methacrylamide] copolymer, polyvinyl alcohol, polyurethane, polyphosphazene, polyacrylate, polyurea, polyamine polyepsilon-caprolactone, and copolymers thereof, wherein the polymer is modified or derivatized to enhance proteolytic resistance, improve circulating half-life, reduce antigenicity, reduce immunogenicity, reduce toxicity, improve solubility, or improve thermal or mechanical stability. In particular embodiments, the shell is biodegradable. In certain embodiments the multivalent polymeric scaffold is cationic and is composed of a polyamide, a polysaccharide, a polyanhydride, poly-L-lysine, a polyacrylamide, a polymethacrylate, a polypeptide, a polyethylene oxide, a polyethylencimine, poly(amidoamine) (PAMAM) or PAMAM (ethylenediamine-EDA).

Another hybrid nanoparticle is a dendrimer-exosome hybrid as described in U.S. application Ser. No. 16/011,922. A dendrimer-exosome hybrid is an exosome loaded with one or more nanoparticle dendrimers. As used herein, exosome refers to small vesicles having a membrane structure that are secreted from various cells. Exosomes have diameters of about 25 to about 150 nm. Exosomes may express markers such as VLA-4, CD162, CXCR4, CD9, CD63, CD81 or a combination thereof. In an embodiment, the exosome is derived from a stem cell or a tumor cell which is isolated from a subject, e.g., a human subject.

In an embodiment, the exosome is derived from a stem cell or a tumor cell which is isolated from a subject, e.g., a human subject.

Stem cells include embryonic stem cells or adult stem cells, preferably, adult stem cells. The adult stem cells may be, without being limited to, mesenchymal stem cells, human tissue-derived mesenchymal stromal cells (mesenchymal stromal cell), human tissue-derived mesenchymal stem cells, multipotent stem cells, or amniotic epithelial cells, preferably, mesenchymal stem cells. The mesenchymal stem cells may be derived from, without being limited to, the umbilical cord, umbilical cord blood, bone marrow, fat, muscle, nerve, skin, amnion, placenta, and the like.

In an embodiment, the stem cell is a mesenchymal stem cell. Mesenchymal stem cells (MSCs) can specifically target inflammatory regions that are frequently found in cancerous regions, i.e., MSC tumor-homing.

In another embodiment, the exosome is isolated from a tumor cell. Tumor cells actively produce, release, and utilize exosomes to promote tumor growth.

Exosomes can be produced by isolating tumor or stem cells from a subject, expanding the tumor or stem cells to provide an expanded cell population, culturing the expanded cell population, and isolating the exosome secreted from the expanded tumor or stem cells. The internal components can be removed from the isolated exosomes to provide so-called ghost exosomes which are essentially empty vessels for loading components such as nanoparticle dendrimers. Exosomes derived from a patient can provide a non-immunogenic nanocarrier shell to the patient, in addition to the features above, allowing an option for personalized medicine.

In order to allow for conjugation of the immune checkpoint inhibitors, in one aspect, the multivalent nanoparticles are modified by reaction with alkyl epoxides, wherein the R group of the epoxide has 1 to 30 carbon atoms. In some embodiments, the alkyl epoxides react with amino groups present on the multivalent nanoparticles to form alkylated multivalent nanoparticles.

Amine groups present on the multivalent nanoparticles provide reactive sites for a variety of amine-based conjugation reactions using coupling linkers that include, but are not limited to, dicyclohexylcarbodiimide, diisopropylcarbodiimide, N-(3-Dimethylaminopropyl)-N′-ethylcarbodiimide, 1,1′-carbonyldiimidazole, N-succinimidyl S-acetylthioacetate, N-succinimidyl-S-acetylthiopropionate, 2-Mercaptoethylamine, sulfosuccinimidyl 4-(N-maleimidomethyl) cyclohexane-1-carboxylate, succinimidyl iodoacetate, succinimidyl 3-(2-pyridyldithio) propionate. In some embodiments, reactive esters are used to link multivalent nanoparticles and other compounds via ester bonds. Examples of the reactive esters include, but are not limited to, N-hydroxysuccinimide ester, N-hydroxy sulfosuccinimide ester, N-y-maleimidobutyryl-oxysulfosuccinimide ester, nitrophenyl ester, tetrafluoro phenyl ester, pentafluorophenyl ester, thiopyridyl ester, thionitrophenyl ester. Preferably, the reactive ester group is an N-hydroxysuccinimide ester.

In an aspect, the peptide may include a linker and/or chemical modifications to facilitate conjugation with the nanoparticle core. Exemplary linkers include amide linker, thioether linkers, maleimidocaproyl linkers, click chemistry linkers, and the like. The peptide may include additional amino acids to facilitate conjugation to the nanoparticle core and/or interaction with the target receptor.

The nanoparticle system comprises a plurality of conjugated therapeutic peptides. The large number of end groups on the multivalent nanoparticle core allows for conjugation of a wide variety of molecules in addition to the therapeutic peptides.

In an aspect, the multivalent nanoparticle core can be associated with, e.g., complexed or conjugated with, one or more of a therapeutic, prophylactic or diagnostic agent. Diagnostic agents include imaging agents. Therapeutic molecules, diagnostic agents, and prophylactic agents may be combined with multivalent nanoparticle core via chemical conjugation, physical encapsulation, and/or electrostatic interaction methods.

In an aspect, a method of making a nanoparticle system comprises contacting the multivalent nanoparticle cores comprising multiple reactive end groups with a composition comprising therapeutic peptides under conditions sufficient to conjugate a plurality of therapeutic peptides to the multivalent nanoparticle cores and provide the nanoparticle system. Exemplary end groups include coupling linkers and reactive epoxides, such as dicyclohexylcarbodiimide, diisopropylcarbodiimide, N-(3-dimethylaminopropyl)-N′-ethylcarbodiimide, 1,1′-carbonyldiimidazole, N-succinimidyl S-acetylthioacetate, N-succinimidyl-S-acetylthiopropionate, 2-Mercaptocthylamine, sulfosuccinimidyl 4-(N-maleimidomethyl) cyclohexane-1-carboxylate, succinimidyl iodoacetate, succinimidyl 3-(2-pyridyldithio) propionate, N-hydroxysuccinimide ester, N-hydroxy sulfosuccinimide ester, N-y-maleimidobutyryl-oxysulfosuccinimide ester, nitrophenyl ester, tetrafluoro phenyl ester, pentafluorophenyl ester, thiopyridyl ester, thionitrophenyl ester, and combinations comprising at least one of the foregoing.

In an embodiment, the multivalent nanoparticle cores comprise two or more different types of reactive end groups to enhance the reactivity and/or specificity of the cores.

In another embodiment, an immunotherapy method comprises administering to the subject, e.g., a human subject, a nanoparticle system as described herein. Exemplary human subjects include cancer patients and patients with immune disorders such as multiple sclerosis and rheumatoid arthritis. The nanoparticles can target the immune system by interacting with T cells, cancer cells and/or antigen presenting cells.

Also included are pharmaceutical compositions comprising the nanoparticle system described herein.

As used herein, “pharmaceutical composition” means therapeutically effective amounts of the nanoparticles together with a pharmaceutically acceptable excipient, such as diluents, preservatives, solubilizers, emulsifiers, and adjuvants. As used herein “pharmaceutically acceptable excipients” are well known to those skilled in the art.

Tablets and capsules for oral administration may be in unit dose form, and may contain conventional excipients such as binding agents, for example syrup, acacia, gelatin, sorbitol, tragacanth, or polyvinyl-pyrrolidone; fillers for example lactose, sugar, maize-starch, calcium phosphate, sorbitol or glycine; tabletting lubricant, for example magnesium stearate, talc, polyethylene glycol or silica; disintegrants for example potato starch, or acceptable wetting agents such as sodium lauryl sulphate. The tablets may be coated according to methods well known in normal pharmaceutical practice. Oral liquid preparations may be in the form of, for example, aqueous or oily suspensions, solutions, emulsions, syrups or elixirs, or may be presented as a dry product for reconstitution with water or other suitable vehicle before use. Such liquid preparations may contain conventional additives such as suspending agents, for example sorbitol, syrup, methyl cellulose, glucose syrup, gelatin hydrogenated edible fats; emulsifying agents, for example lecithin, sorbitan monooleate, or acacia; non-aqueous vehicles (which may include edible oils), for example almond oil, fractionated coconut oil, oily esters such as glycerine, propylene glycol, or ethyl alcohol; preservatives, for example methyl or propyl p-hydroxybenzoate or sorbic acid, and if desired conventional flavoring or coloring agents.

For topical application to the skin, the drug may be made up into a cream, lotion or ointment. Cream or ointment formulations which may be used for the drug are conventional formulations well known in the art. Topical administration includes transdermal formulations such as patches.

For topical application to the eye, the inhibitor may be made up into a solution or suspension in a suitable sterile aqueous or non-aqueous vehicle. Additives, for instance buffers such as sodium metabisulphite or disodium edeate; preservatives including bactericidal and fungicidal agents such as phenyl mercuric acetate or nitrate, benzalkonium chloride or chlorhexidine, and thickening agents such as hypromellose may also be included.

The active ingredient may also be administered parenterally in a sterile medium, either subcutaneously, or intravenously, or intramuscularly, or intrasternally, or by infusion techniques, in the form of sterile injectable aqueous or oleaginous suspensions. Depending on the vehicle and concentration used, the drug can either be suspended or dissolved in the vehicle. Advantageously, adjuvants such as a local anesthetics, preservative and buffering agents can be dissolved in the vehicle.

Pharmaceutical compositions may conveniently be presented in unit dosage form and may be prepared by any of the methods well known in the art of pharmacy. The term “unit dosage” or “unit dose” means a predetermined amount of the active ingredient sufficient to be effective for treating an indicated activity or condition. Making each type of pharmaceutical composition includes the step of bringing the active compound into association with a carrier and one or more optional accessory ingredients. In general, the formulations are prepared by uniformly and intimately bringing the active compound into association with a liquid or solid carrier and then, if necessary, shaping the product into the desired unit dosage form.

The nanoparticle compositions described herein can be co-administered with a chemotherapeutic agent. Chemotherapeutic agents include, but are not limited to, the following classes: alkylating agents, antimetabolites, anthracyclines, plant alkaloids, topoisomerase inhibitors, monoclonal antibodies, and other anti-tumor agents. In addition to the chemotherapeutic drugs described above, namely doxorubicin, paclitaxel, other suitable chemotherapy drugs include tyrosine kinase inhibitor imatinib mesylate (Gleeve® or Glivec®), cisplatin, carboplatin, oxaliplatin, mechloethamine, cyclophosphamide, chlorambucil, azathioprine, mercaptopurine, pyrimidine, vincristine, vinblastine, vinorelbine, vindesine, podophyllotoxin (LO1CB), etoposide, docetaxel, topoisomerase inhibitors (LO1CB and L01XX) irinotecan, topotecan, amsacrine, etoposide, etoposide phosphate, teniposide, dactinomycin, lonidamine, and monoclonal antibodies, such as trastuzumab (Herceptin®), cetuximab, bevacizumab and rituximab (Rituxan®), among others.

Other examples of therapeutic agents include, but are not limited to, antimicrobial agents, analgesics, anti-inflammatory agents, and others. Antibiotics can be incorporated into the particle, such as vancomycin, which is frequently used to treat infections, including those due to methicillin resistant Staph aureus (MRSA). The particle optionally includes cyclosporin, a lipophilic drug that is an immunosuppressant agent, widely used post-allogeneic organ transplant to reduce the activity of the patient's immune system and the risk of organ rejection (marketed by Novartis under the brand names Sandimmune® and Neoral®). Particles comprising cyclosporine can be used in topical emulsions for treating keratoconjunctivitis sicca, as well. In this regard, particles with multifunctional surface domains incorporating such drugs can be designed to deliver equivalent dosages of the various drugs directly to the cancer cells, thus potentially minimizing the amount delivered generally to the patient and minimizing collateral damage to other tissues.

Therapeutic agents also include therapeutic nucleic acids such as gene-silencing agents, gene-regulating agents, antisense agents, peptide nucleic acid agents, ribozyme agents, RNA agents, and DNA agents. Nucleic acid therapeutic agents include single stranded or double-stranded RNA or DNA, specifically RNA, such as triplex oligonucleotides, ribozymes, aptamers, small interfering RNA including siRNA (short interfering RNA) and shRNA (short hairpin RNA), antisense RNA, microRNAs (miRNAs), or a portion thereof, or an analog or mimetic thereof, that is capable of reducing or inhibiting the expression of a target gene or sequence. Inhibitory nucleic acids can act by, for example, mediating the degradation or inhibiting the translation of mRNAs which are complementary to the interfering RNA sequence.

The compositions and methods described herein are applicable to all cancers including solid tumor cancers, e.g., those of the breast, prostate, ovaries, lungs and brain, and liquid cancers such as leukemias and lymphomas.

The methods described herein can be further combined with additional cancer therapies such as radiation therapy, chemotherapy, surgery, and combinations thereof.

The invention is further illustrated by the following non-limiting examples.

### EXAMPLES

### Materials and Methods

### Phage Display

To identify high-affinity TIGIT binding peptide candidates, the Ph.D™-12 Phage Display Peptide Library Kit v2 (New England BioLabs) was used for surface panning experiments. Fresh glycerol stocks of E. coli (ER2738) were streaked onto LB (Lennox L) plates with Tet (tetracycline, 20 μg/mL) and incubated ON at 37° C. The next day, the stock culture was wrapped in Parafilm™ and stored in the dark at 4° C. for up to 1 month.

Once all stocks and solutions were ready, surfaces were prepared with mouse TIGIT protein (Acro Biosystems) [10 μg/mL of protein in 0.1 M sodium bicarbonate (NaHCO₃)] by coating a sterile PS petri dish and incubating ON at 4° C. with gentle agitation. Surface panning was then carried out using a combinatorial library of random 12-mer peptides fused to a minor coat protein (pIII) of M13KE phage. With the surface prepared, LB media with Tet was inoculated with ER2738 and incubated ON at 37° C. with vigorous shaking. The coated surface was then blocked [0.1 M NaHCO₃ (pH 8.6), 5 mg/mL BSA, and 0.02% sodium azide (NaN3)] for 1 hr at 4° C., washed with TBST (TBS+0.1% [v/v] Tween-20), and then incubated with a 100-fold representation of the phage library (diluted in TBST) for 1 hr at RT.

After incubation, unbound phage were removed, and the surface was again washed with TBST. Bound phage were eluted [0.2 M Glycine-HCl (pH 2.2) and 1 mg/mL BSA] for 20 min at RT, neutralized [1 M Tris-HCl (pH 9)] and stored at 4° C. until ready for use. The phage were added to a diluted culture (1:100) of ER2738 in LB medium and incubated at 37° C. with vigorous shaking for 4.5 hrs. The culture was then centrifuged at 4,500×gravity (g)/12 min at 4° C. The supernatant was transferred to a new tube, re-centrifuged, and mixed 1:4 with PEG/NaCl buffer (25 mM PEG-8000, 2.5 M NaCl) to precipitate phage ON at 4° C.

Following precipitation, the phage were centrifuged (4500×g/40 min/4° C.). The supernatant was decanted, and TBS was added to the pellet and vortexed. The suspension was then again centrifuged (20000×g/1 min/4° C.) to pellet residual cells. The supernatant (amplified eluate) was transferred to a fresh tube, reprecipitated on ice for 1 hr, and again resuspended in TBS. The amplified eluate was tittered, and serial dilutions were prepared in LB medium, mixed with diluted (1:1 in LB medium) ER2738 culture, and incubated at RT. The dilutions were then added to pre-warmed agar, vortexed, and poured onto pre-warmed LB plates with IPTG (Isopropyl β-D-1-thiogalactopyranoside) and Xgal (5-bromo-4-chloro-3-indolyl-B-D-galacto-pyranoside), and incubated ON at 37° C.

This process was repeated for multiple rounds of panning to allow for sufficient enrichment of high-affinity phages-using 1E11 plaque forming units (PFU) of amplified eluate from the previous round of panning for incubation. Once completed, individual plaques from the LB plates with IPTG and Xgal were amplified in separate ER2738 cultures. Once amplified, the cultures were centrifuged (20000×g/1 min/4° C.), transferred to fresh tubes and re-precipitated. Precipitates were again centrifuged (20000×g/10 min/4° C.), and the pellet was resuspended in iodide buffer [10 mM Tris-HCl (pH 8.0), 1 mM EDTA, and 4 M sodium iodide (NaI)]. This suspension was then mixed with 100% EtOH, incubated for 20 min at RT, and again centrifuged (20000×g/10 min/4° C.) to obtain a pellet. This pellet was resuspended in 70% EtOH and centrifuged (20000×g/10 min/4° C.), dried under vacuum, and resuspended in TE buffer [10 mM Tris-HCl (pH 8.0) and 0.1 mM EDTA]. The concentration and A280/260 ratio were determined for all samples, following which they were prepared with 0.5 μg template and 1 pmol primer (−96gIII) in TE buffer and submitted to the UW-Madison Biotechnology Center for Sanger sequencing. Once the reads were obtained, they were matched based on the primer (corresponding to the anticodon strand of the gIII region of the template), and the library insert was located. The insert was then decoded and translated into amino acid sequences.

### Synthesis and Characterization of TIGIT-Binding Peptides

The TIGIT binding peptides were synthesized using standard Fmoc chemistry on a Liberty Blue™ 2.0 microwave synthesis system (CEM) using a rink amide scaffold (0.1 mM). Deprotection was performed with piperidine (10%), and coupling reactions used a 5-fold excess of amino acids (0.2 M) with DIC (1 M) and Oxyma Pure® (1 M) in DMF. The coupling temperature was set at 90° C./2 min for all amino acids, except for His, (50° C./10 min) and Arg (double coupling). If necessary, peptides were acetylated with AA (10% in DMF). After synthesis, peptides were deprotected and cleaved from the resin using a cleavage cocktail (95% TFA, 2.5% EDT, and 2.5% thioanisole), then dried and precipitated using TBME. The precipitate was then dissolved in an ACN/H₂O mixture, filtered (0.45 μm), and dried. The concentrate was fractionated using reverse-phase HPLC (Agilent 1260 Infinity II) with a C18 semi-preparative column against a mobile phase gradient elution of ACN/H₂O with 0.1% TFA to obtain purified peptide. The MW of the purified peptides was confirmed by MALDI-TOF MS (Shimadzu AXIMA linear MALDI TOF mass spectrometer with a CovalX™ high-mass system) after mixing the sample (1:1) with CHCA matrix. Additional purification was performed if necessary.

BLI analysis was performed using the Forte Octet® Red 96 (Sartorius) with Octet® Amine Reactive Second-Generation (AR2G) Biosensors. Briefly, biosensors were hydrated (600 s) and equilibrated (60 s) in H₂O (60 s), activated with EDC/Sulfo-NHS (400 mM EDC and 200 mM NHS) solution (300 s), immobilized with recombinant mouse TIGIT Fc Chimera protein (BioTechne, 20 μg/mL) [in 10 mM sodium acetate (pH 5.0)] (600 s), quenched (1 M Ethanolamine) (300 s), and washed in buffer (PBS with 0.02% Tween-20, 0.1% BSA, and 0.05% NaN3) to achieve a baseline (120 s). Once at baseline, the biosensors were reacted with samples to obtain association and dissociation curves (900 s). The data was then processed using Octet® BLI Discovery Software for reference subtraction, inter-step correction, and Savitzky-Golay filtering. The processed data was exported and evaluated through curve fitting, assuming 1:1 Langmurian kinetics. Following processing, data was exported and evaluated through curve fitting. Data was then loaded into GraphPad Prism version 10.2.3 (GraphPad Software, Boston, Massachusetts USA) for analysis.

AFM was used to record unbinding events between the probe-immobilized recombinant TIGIT proteins and surface-immobilized peptides, DPCs, and controls. To prepare the probes, PNP-TR-Au-20 AFM probes (Nano World) were incubated with a mixture of 7500 carboxyl-PEG-thiol (0.05 mg/mL) and 5000 methoxy-PEG-thiol (5 mg/mL) (JenKem) in conjugation buffer [100 mM sodium phosphate dibasic (Na₂HPO₄) and 150 mM NaCl, PH 8] ON at RT. Subsequently, the probes were washed with ddH₂O and reacted with EDC/NHS (1.8 mg/mL and 2.8 mg/mL in H₂O, respectively) for 30 min at RT. Following this incubation period, the probes were reacted with recombinant mouse TIGIT Fc Chimera protein (BioTechne, 20 μg/mL) for a minimum of 1 hr at RT. The spring constant of the probes was measured to be 37.6-51.5 pN/nm (via the thermal noise method).

To prepare the surfaces, epoxy-functionalized microscope slides (Tekdon) were washed with ddH₂O and treated with 5000 amine-PEG-amine (JenKem) in ddH₂O ON. DPCs were dissolved in Na₂HPO₄ buffer along with EDC and NHS and allowed to react with the surface for a minimum of 1 hr at RT. Peptide and mAb control surfaces were prepared in parallel with 2 mg/mL 5000 carboxyl-PEG-amine (JenKem), activated with EDC/NHS in ddH₂O for 30 min, then reacted with normalized concentrations of peptide (peptides/dendrimer) or mAb (molarity) for a minimum of 1 hr at RT.

Force spectroscopy was performed using an Asylum Infinity™ Biosystem (Oxford Instruments). Both surfaces and probes were submerged in 2% BSA in PBS. Force curves were obtained with a 3 μm approach length at 1 μm/s with 5 s dwell time. At least 30 curves were collected at 5 different areas. The curves were analyzed using a custom-written code in R (version 4.1.2) using the IgorR package. Data were smoothed using a median window filter spanning the equivalent of 5 nm in z. The force curve was corrected to a line fit to the retraction curve away from the surface. Maximum force was defined as the maximum deflection multiplied by the cantilever spring constant. The work required to separate the probe from the surface was defined as the integral (area) of the Force-extension curve. Individual rupture events were defined as abrupt reductions in force (>50 pN in magnitude, at a rate exceeding 50 pN/ms).

### Example 1: Development of High-Affinity DPC T-Cell Immunoreceptors with Ig and ITIM Domains (TIGIT) Binding Peptides

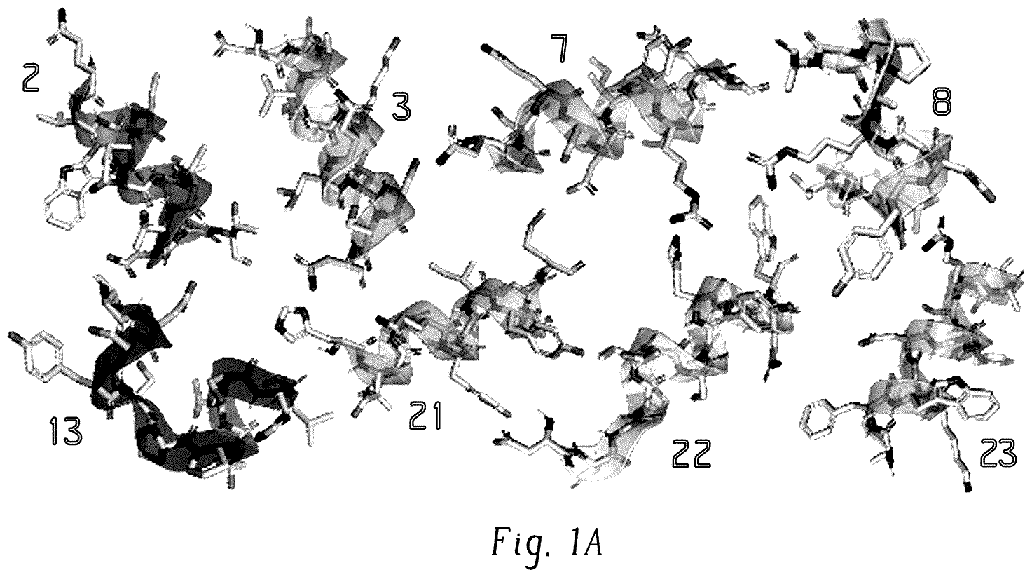

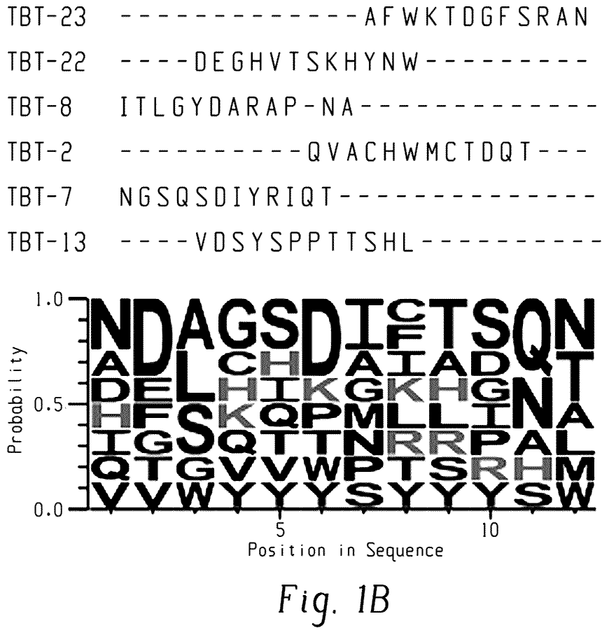

Phage display was employed to identify several highly selective sequences (FIG. 1A, B) that bind to the mouse ortholog of TIGIT (T cell immunoreceptor with Ig and ITIM domains)—an inhibitory receptor expressed on lymphocytes that has been identified as a potential therapeutic target to enhance anti-tumor immune responses. Specifically, the inserts of 24 localizing phage from a library pool were sequenced after 4 rounds of subtractive screening.

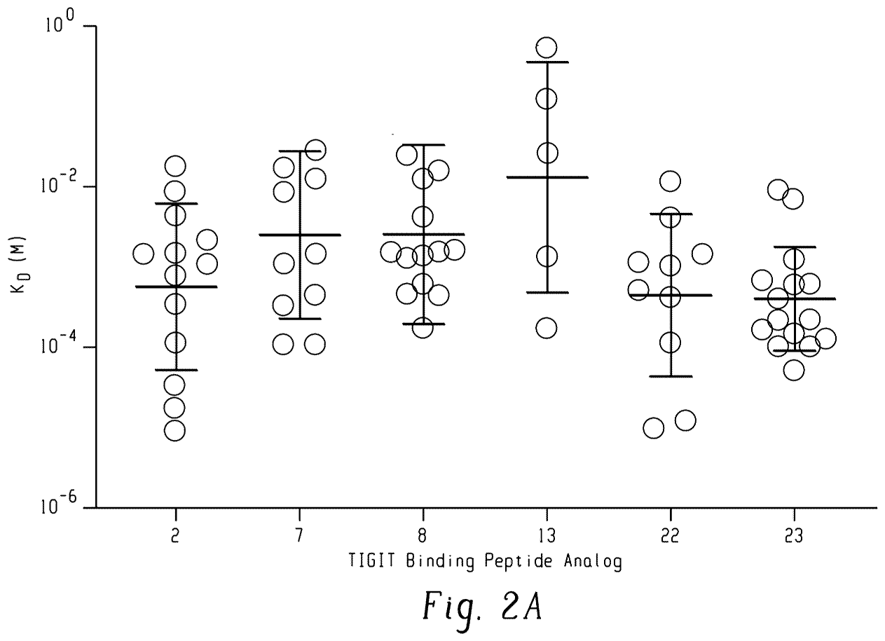

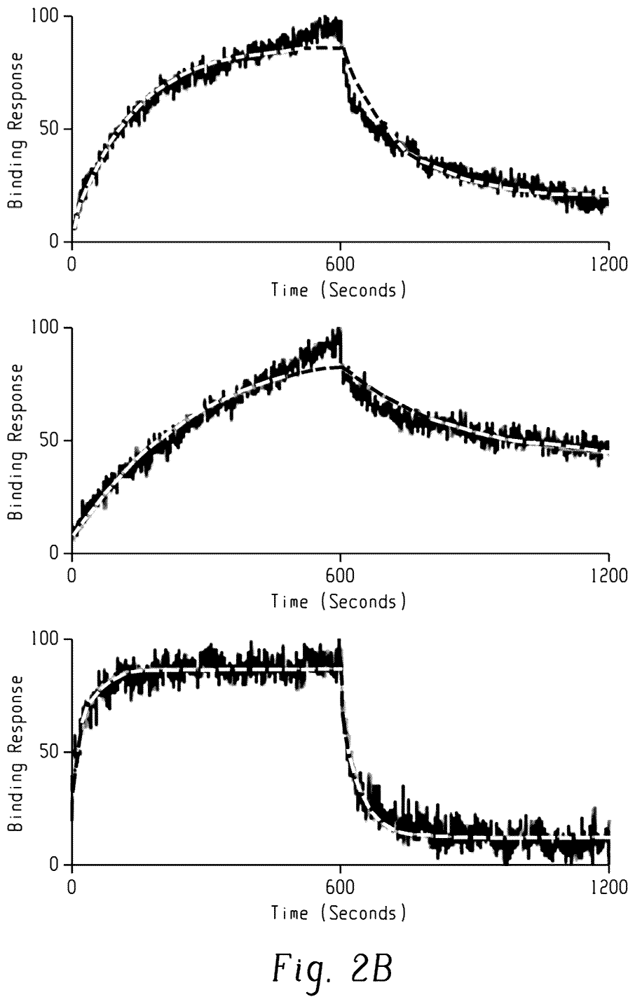

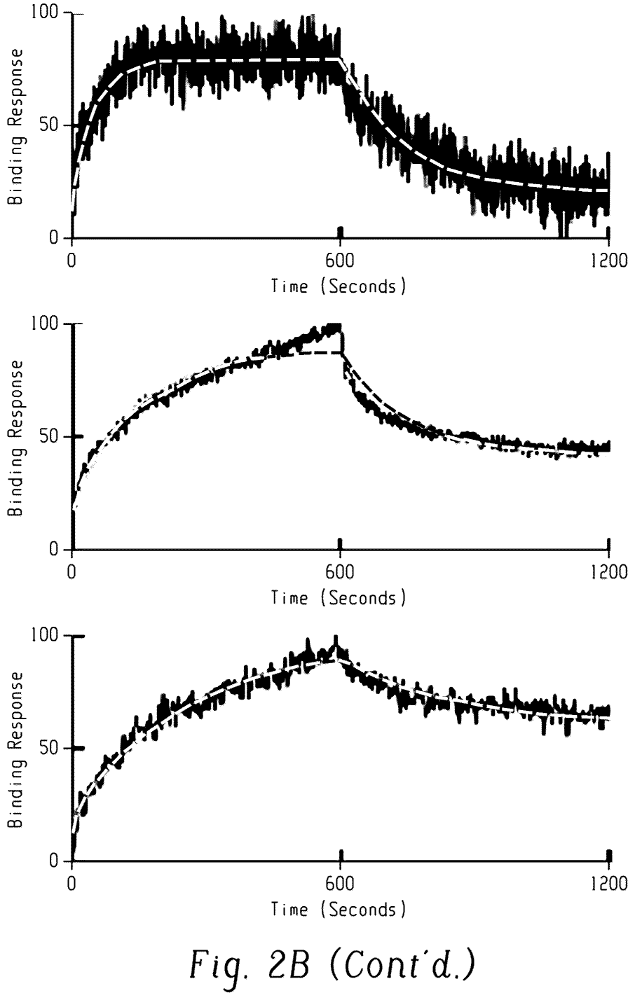

The peptide candidates were synthesized (solid phase peptide synthesis, SPPS) using a standard Fmoc chemistry, and subsequently deprotected, concentrated, and purified. Following the confirmation of a successful synthesis, the peptides' relative binding affinity was analyzed using biolayer interferometry (BLI) (FIG. 2A, B, Table 2).

TABLE 2
Kinetic parameters from BLI experiments

| Sample | kₒₙ (M⁻¹ s⁻¹) | k_{off} (s⁻¹) | K_{D} (M) |
|---|---|---|---|
| 2 | 8.83E1 ± 3.53E1 | 4.07E−3 ± 8.76E−4 | 4.61E−5 ± 2.09E−5 |
| 7 | 4.02E1 ± 2.07E1 | 1.39E−2 ± 3.77E−3 | 3.45E−4 ± 2.00E−4 |
| 8 | 1.32E2 ± 1.16E2 | 1.50E−2 ± 3.85E−3 | 1.14E−4 ± 1.04E−4 |
| 13 | 1.77E2 ± 1.472 | 2.11E−2 ± 7.27E−3 | 1.20E−4 ± 1.08E−4 |
| 22 | 1.86E1 ± 6.59E0 | 1.69E−2 ± 7.66E−3 | 9.09E−4 ± 5.24E−4 |
| 23 | 2.07E1 ± 1.03E1 | 4.20E−3 ± 8.10E−4 | 2.03E−4 ± 1.09E−4 |
| M1 | 7.57E2 ± 5.74E2 | 7.50E−3 ± 3.41E−3 | 9.90E−6 ± 8.75E−6 |

Docking simulations were also performed. Specifically, HPEPDOCK was used, a hierarchical algorithm for blind and flexible peptide docking that performs fast modeling of peptide conformations and subsequent global sampling of binding orientations. This algorithm accounts for peptide flexibility by generating an ensemble of peptide conformations, which are then globally docked against the whole protein. When tested against the TIGIT receptor (P86176), the docking scores aligned with the kinetic values obtained from BLI. Analogs 2, 22, and 23 showed enhanced binding responses with correspondingly lower docking scores, indicating stronger and more stable interactions with the protein and, thus, higher binding affinities.

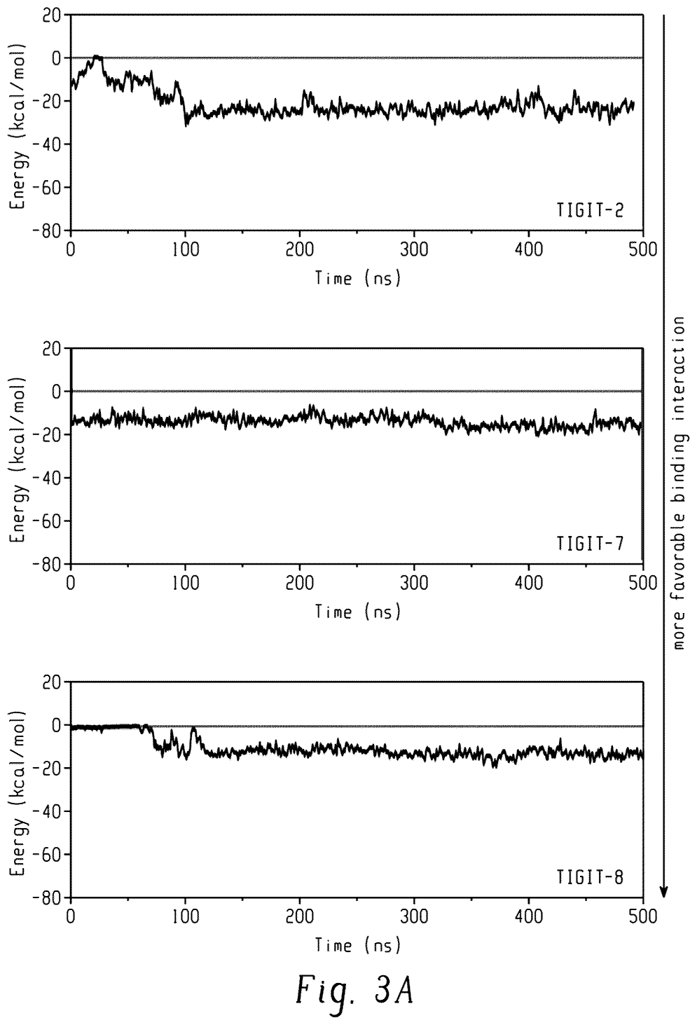

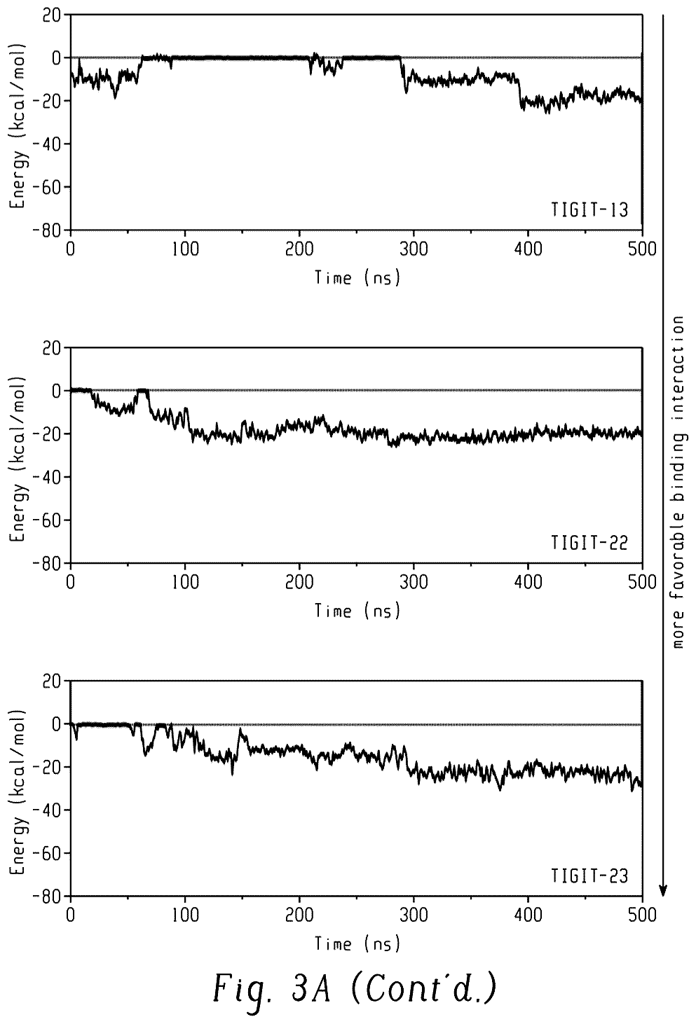

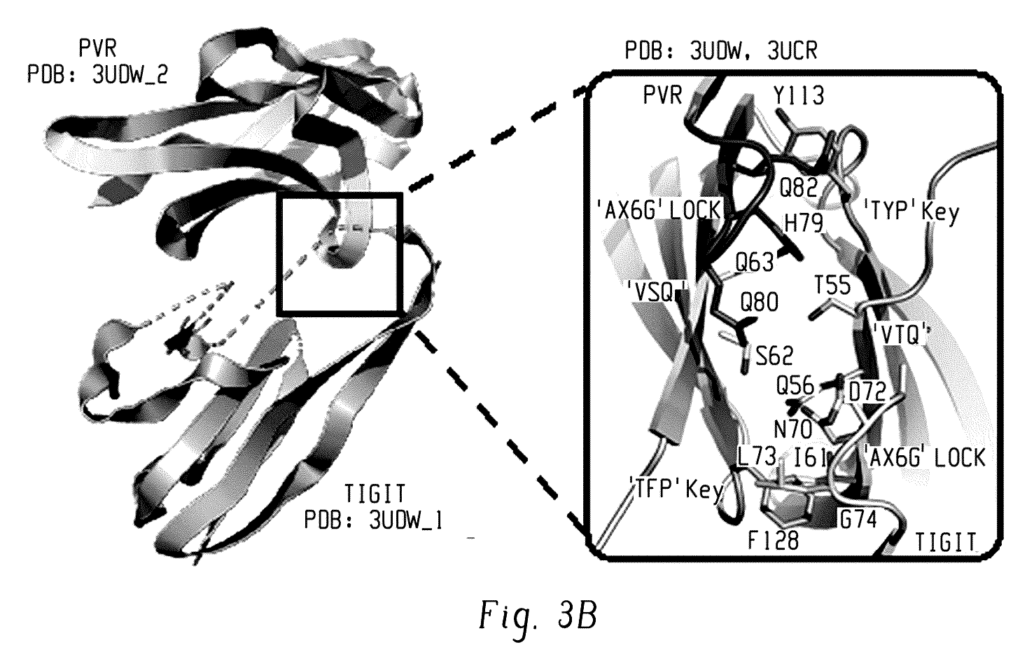

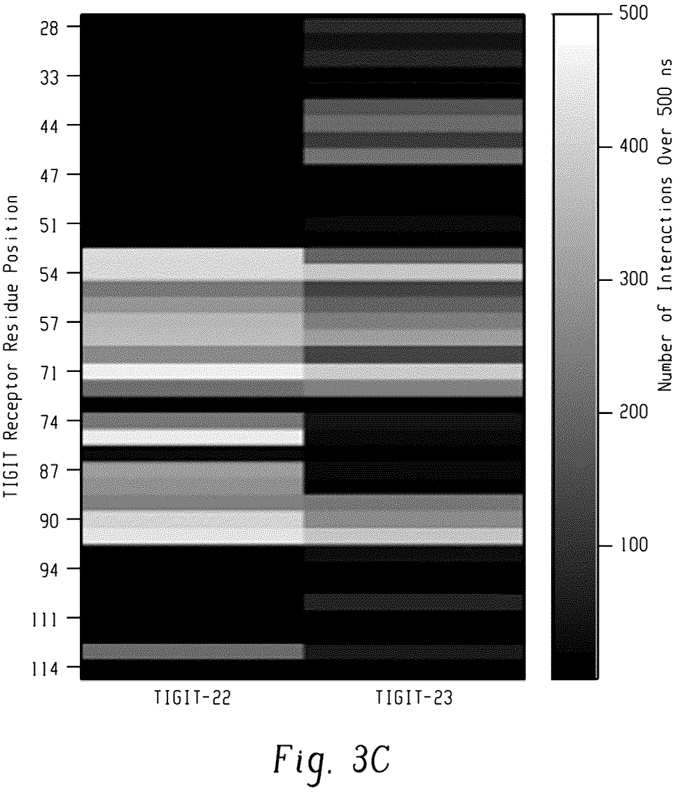

Subsequent molecular dynamics (MD) simulations (NAMD, CHARMM force field) revealed several conserved regions of interactions in both the protein receptor and the binding peptides. We hypothesize that these regions drive favorable binding interactions and enhance the binding affinity of the peptide inhibitors (FIG. 3A-C). Specifically, peptide/receptor interactions were analyzed using Visual Molecular Dynamics and scalable molecular dynamics with an NAMD package using CHARMM general and protein forcefields. The systems were equilibrated using 500 nanoseconds (ns) production molecular dynamics runs with no restraints. The peak interaction energies observed from the simulations are detailed in Table 3. Initially, the focus was on identifying the most common interaction sites among all peptides, focusing on distinct and shared binding pockets; given the length of the analogs (12-mers), exhaustive exploration of all predicted conformations and biological behaviors would be impractical as there are too many degrees of freedom. Notably, analog 2 exhibits fast association kinetics but quickly destabilizes and dissociates from the receptor. In contrast, analogs 22 and 23 display slower association rates, but form stable bonds with slower dissociation rates, resulting in enhanced interaction energies. These results corroborate earlier BLI and docking data, prompting a closer examination of these analogs.

TABLE 3
Average interaction energies as determined by in silico analysis

| | Average interaction energy | |
|---|---|---|
| Analog | (kcal/mol) | Standard error |
| 2 | −17.5524 | 4.6434 |
| 7 | −8.1709 | 3.3329 |
| 8 | −8.0001 | 2.3832 |
| 13 | −8.7570 | 2.1616 |
| 22 | −11.2679 | 3.6189 |
| 23 | −10.7294 | 3.8583 |
| Mutant 1 | −78.2098 | 0.6276 |
| TT | −53.642 | 7.35369 |
| TP1 | −66.239 | 5.62619 |
| TP2 | −63.0632 | 5.79207 |
| TP3 | −63.9696 | 6.64004 |

In the case of analog 22, Gly90 acts as an anchor due to electrostatic charges, facilitating prolonged retention time at the receptor site. Furthermore, analog 23 exhibited specific interactions with TIGIT residues 42-46 (Ile42, Gln43, His44, Leu45, and Ser46), demonstrating that this region forms a small cleft capable of accommodating small amino acids. Simulations indicated that this cleft significantly contributes to favorable binding interactions.

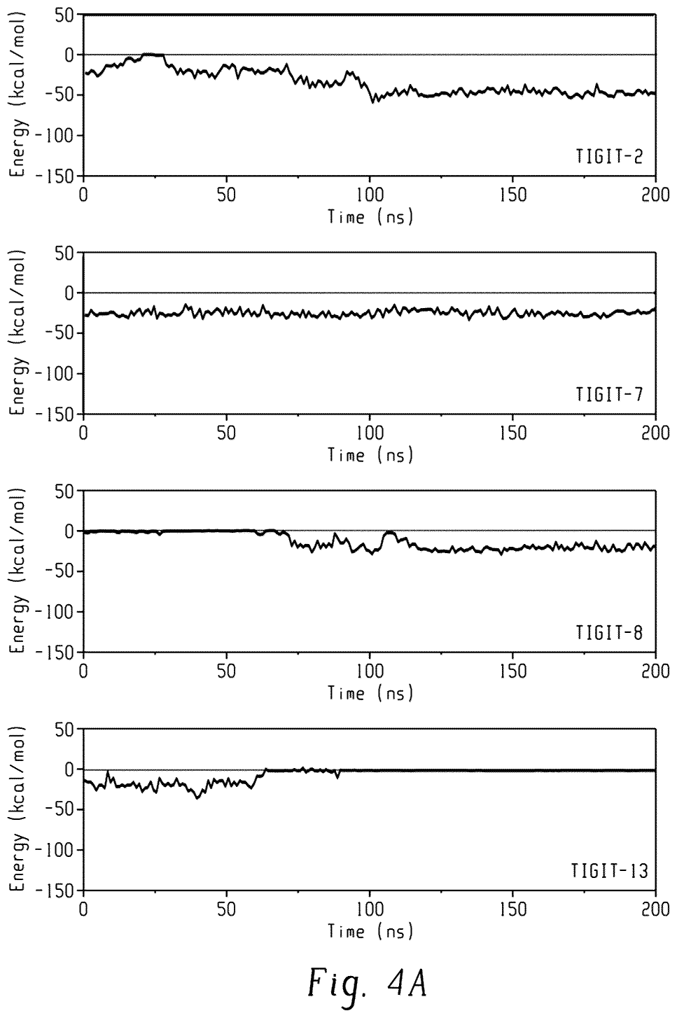

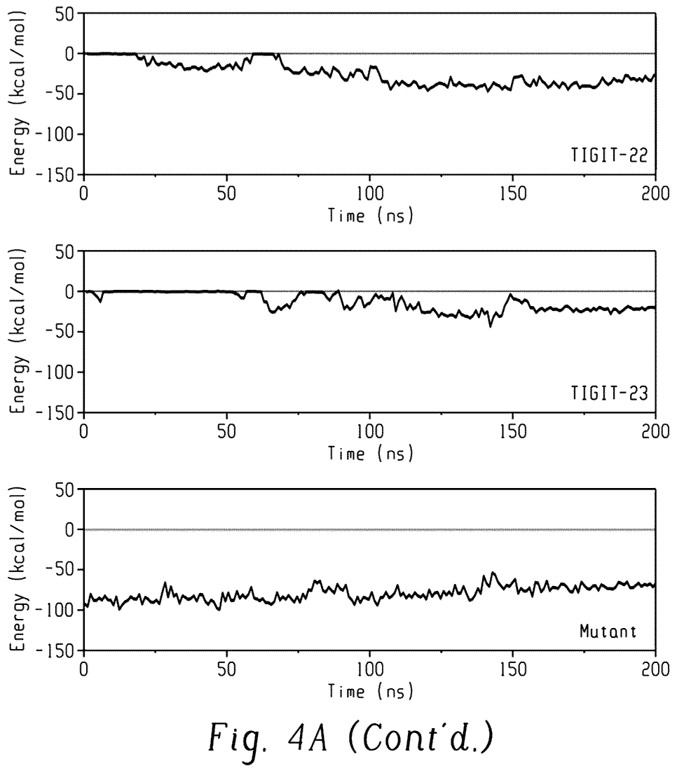

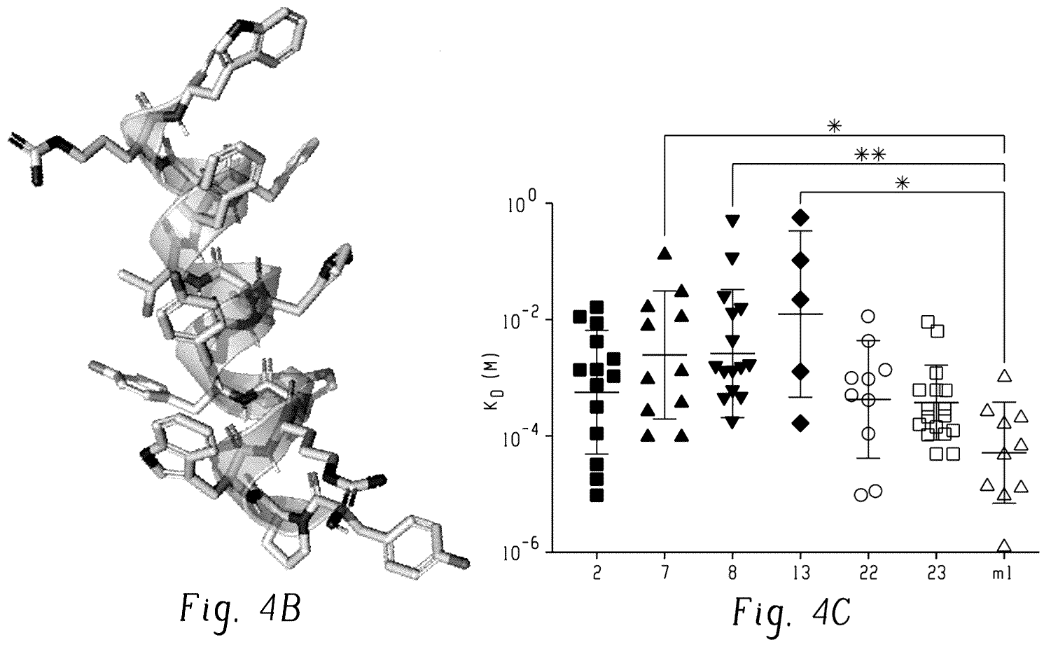

Next, the experimental kinetic parameters (FIG. 2A, B) were incorporated with adaptive evolution computational modeling to identify an optimized mutant analog for dendrimer conjugation. A highly efficient and very fast MD-based approach was identified that uses the initially identified conservative binding regions in the therapeutic peptides and protein receptors to dramatically optimize the mutant peptide. This mutant analog was again synthesized, purified, and analyzed using BLI (FIG. 4A-C).

Specifically, random mutations were introduced across peptide templates derived from experimental data. Subsequently, short molecular dynamics simulations of the mutated peptides interacting with the TIGIT receptor were conducted, followed by MC sampling using a Metropolis criterion to accept or reject mutations based on changes in the binding interaction free energy. The mutation/selection cycle was iteratively repeated until a mutant peptide with a satisfactory predicted binding affinity was identified.

The developed mutant peptide (YPWRWYYHVTFFCRW; SEQ ID NO: 9) was found to have two orders of magnitude enhancement in binding affinity towards the TIGIT receptor relative to its analog counterparts. In addition, the mutant peptide alone was comparable to a monoclonal TIGIT-targeting antibody in terms of binding affinity, with subsequently synthesized DPCs demonstrating even greater avidity than both. Previous work has demonstrated that such an enhancement translates into in vitro selectivity and in vivo efficacy.

Following in silico validation, the peptide was synthesized using microwave-assisted SPPS. Subsequently, it was deprotected, cleaved from the resin, and concentrated for fractionation using reverse-phase HPLC. Similar to the previous analogs, the peptide was purified, and its MW was confirmed using MALDI-TOF mass spectrometry.

The mutant peptide's kinetic parameters were probed using BLI to collect interaction curves, which were later analyzed via curve fitting, following the assumption of conformity to 1:1 Langmurian kinetics. This analysis corroborated the in silico simulations, revealing that the mutant peptide exhibited a K_{D} in the micromolar (μM) range, whereas the other analogs showed values in the millimolar (mM) range. Scrambled versions of selected analogs (22, 23, and the mutant) were also synthesized, and the binding kinetics were experimentally evaluated; significant differences in K_{D} values (data not shown) were observed between these peptides and their corresponding scrambled versions, highlighting the specificity of the sequences.

### Example 2: Dendrimers Functionalized with TIGIT-Binding Peptides

Using potentiometric titration, the mean number of G7 PAMAM dendrimer end groups was determined to be approximately 439, compared to the theoretical value of 512 primary amine groups. After determining the mean number of end groups, the dendrimers underwent acetylation, targeting approximately 70% of primary amines to provide a more neutral charge and control the number of peptides per dendrimer. Subsequently, the remaining free amines were carboxylated to facilitate EDC/NHS chemistry for peptide conjugation and to further reduce the dendrimers' cationic surface charge. Following carboxylation, EDC/NHS chemistry was subsequently employed for peptide conjugation. To characterize the DPCs, NMR and HPLC analyses were performed. 2D NMR (diffusion ordered spectroscopy, DOSY) was first used to determine the number of peptides per dendrimer, as it provides insights into the physicochemical properties of molecules by studying their molecular diffusion in solution. (data not shown) Using an HPC analysis, number of peptides per dendrimer calculated was 47.26, 40.59, and 36.24 (analogs 22, 23, and the mutant TIGIT-binding peptide SEQ ID NO: 9).

AFM was used to examine the binding interactions of the analogs of interest, focusing on recording unbinding events between probe-immobilized recombinant TIGIT proteins and surface-immobilized DPCs and controls. AFM demonstrated the potent affinity of the mutant peptide to the TIGIT receptor, evidenced by the significant enhancement in maximum adhesion force relative to the other analogs' corresponding DPCs as shown in Table 4.

Table 4: Average retract work and maximum adhesion forces measured for all analogs and dendrimer-peptide conjugates of interest (±SEM).

### Example 3: In Vitro Characterization of DPCs: Effects on Natural Killer-Cell Binding

| | | Average Retract | |
|---|---|---|---|
| Analog | Sample | Work (pN · nm) | Max Force (pN) |
| 22 | Peptide | 6263 ± 1030 | 90.39 ± 13.39 |
| | DPC | 27300 ± 4561 | 405.8 ± 56.65 |
| 23 | Peptide | 4373 ± 857 | 94.46 ± 12.62 |
| | DPC | 8140 ± 1564 | 163.7 ± 29.74 |
| M1 | Peptide | 2950 ± 553 | 78.14 ± 11.17 |
| | DPC | 52594 ± 6222 | 591.9 ± 43.64 |

### Efficiency and Mediated Cytotoxicity

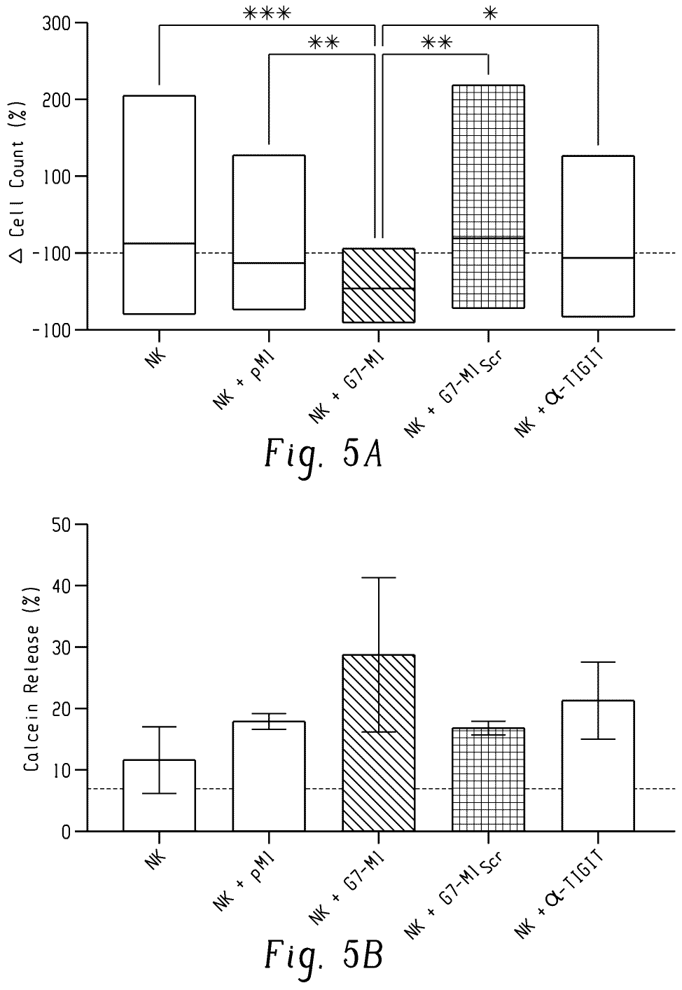

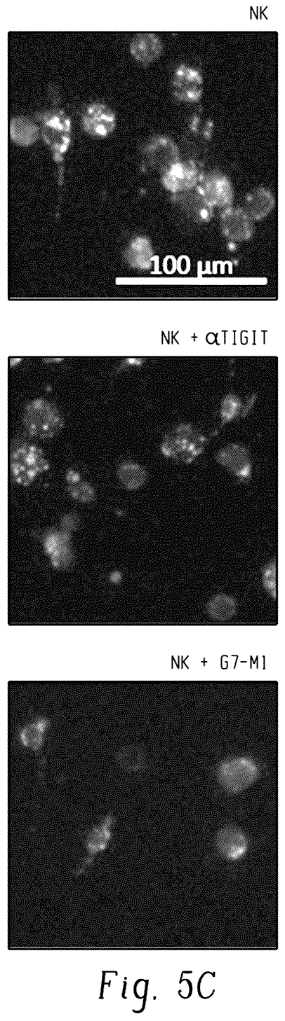

The efficacy of the DPCs in modulating the TIGIT/CD155 axis was evaluated using an in vitro co-culture model using primary NK and murine osteosarcoma (K7M2) cells. K7M2 cells were selected due to their demonstrated utility in assessing CD155 inhibitors, relative CD155 expression (determined using western blotting. As shown in FIG. 5A, when analyzing the null (untreated) control, NK cells exhibited an exhausted phenotype with reduced cytotoxic capabilities. The addition of an inhibitor (such as mAbs, peptides, or DPCs) appeared to alleviate NK cell exhaustion, with the exception of the scrambled DPC group, which showed outcomes similar to the null group. Notably, no significant difference was observed between the mAb and free mutant peptide; however, the inclusion of the dendrimer significantly enhanced lysis efficiency (≥450% relative to null), likely due to its potent inhibition of the TIGIT/CD155 axis through multivalent binding interactions.

As shown in FIG. 5B, when examining the percent of Calcein release, wells were normalized against maximum lysis wells (K7M2 incubated with 2% Triton in media, resulting in complete lysis). These results corroborate previous findings, demonstrating that NK cells pre-treated with mutant DPCs remained activated and efficiently lysed tumor cells compared to the other test groups.

### Example 4: Design of Additional TIGIT Peptides

In the TIGIT-TIGIT or TIGIT-PVR interactions, the region of TIGIT that needs to be inhibited was identified. A sequential screening of residues was used, where optimized residues were obtained based on the protein surface. To further improve the peptides, some of their residues that bind to the protein were manually optimized and residues that were too hydrophobic were replaced.

The peptide N-Term-ARG-THR-ARG-TRP-TYR-ARG-GLY-VAL-THR-MET-GLY-GLU-TRP-C-Term (SEQ ID NO: 11) was designed for the TIGIT-TIGIT interface. Both TIGIT-TIGIT dimerization and the TIGIT-PVR complex can be inhibited. The first peptide was designed for the interface of TIGIT facing the other TIGIT protein. The other peptides were designed for the interface between TIGIT-PVR. The binding energy was-61.29 kcal/mol and the MMGB-SA energy was-53.642 kcal/mol (250 ns).

The peptide N-Term-GLN-TYR-PHE-PRO-GLU-ASN-ARG-VAL-MET-GLU-ARG-ALA-TRP-ARG-C-Term (SEQ ID NO: 12) was designed for the TIGIT-PVR interface. The MMGB-SA energy after 300 ns was-66.239 kcal/mol.

The peptide N-Term-GLN-TYR-PHE-PRO-MET-THR-ARG-VAL-MET-GLU-ARG-ALA-TRP-ARG-C-Term (SEQ ID NO: 13) was designed for the TIGIT-PVR interface. The MMGB-SA energy after 300 ns was-63.0632 kcal/mol.

The peptide N-Term-QYFPENRCMERAWR-C-Term (SEQ ID NO: 14) was designed for the TIGIT-PVR interface. The MMGB-SA energy after 300 ns was-63.9696 kcal/mol.

The use of the terms “a” and “an” and “the” and similar referents (especially in the context of the following claims) are to be construed to cover both the singular and the plural, unless otherwise indicated herein or clearly contradicted by context. The terms first, second etc. as used herein are not meant to denote any particular ordering, but simply for convenience to denote a plurality of, for example, layers. The terms “comprising”, “having”, “including”, and “containing” are to be construed as open-ended terms (i.e., meaning “including, but not limited to”) unless otherwise noted. Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable. All methods described herein can be performed in a suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the invention as used herein.

While the invention has been described with reference to an exemplary embodiment, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims. Any combination of the above-described elements in all possible variations thereof is encompassed by the invention unless otherwise indicated herein or otherwise clearly contradicted by context.

1. A T cell immunoreceptor with Ig and ITIM domains (TIGIT)-binding peptide comprising the amino acid sequence of any of SEQ ID NOs. 1-14, or a TIGIT-binding peptide comprising the amino acid sequence of any of SEQ ID NOs. 1-14 with 1-3 amino acid substitutions, wherein the TIGIT-binding peptide binds mouse TIGIT (SEQ ID NO: 15) or human TIGIT (SEQ ID NO: 16) with a dissociation constant of 10⁻³ M or less.

2. The peptide of claim 1, conjugated to a diagnostic or imaging agent.

3. The peptide of claim 1, wherein the peptide is circularized.

4. A nanoparticle system comprising, a multivalent nanoparticle core comprising a plurality of peptides of claim 1 conjugated thereto.

5. The nanoparticle system of claim 4, wherein the multivalent nanoparticle core comprises a hyperbranched polymer, a dendrimer, a dendron, a hybrid nanoparticle, or a micelle.

6. The nanoparticle system of claim 5, wherein the micelle comprises an amphiphilic dendron-coil, or a dendrimer-exosome hybrid.

7. The nanoparticle system of claim 5, wherein the hybrid nanoparticle comprises a multivalent polymeric scaffold nanoparticle core with the peptide covalently attached thereto; and an outer shell encapsulating the polymeric scaffold nanoparticle core, wherein the outer shell comprises a liposome or a polymeric shell.

8. The nanoparticle system of claim 5, wherein the dendrimer is a poly(amido-amine) (PAMAM) dendrimer, a polyester dendrimer, a polypropyleneimine (PPI) dendrimer, a diaminobutane amine polypropylenimine tetramine (DAB-Am 4) dendrimer, a polypropylamine (POPAM) dendrimer, a polylysine dendrimer, a polyester dendrimer, an iptycene dendrimer, an aliphatic poly(ether) dendrimer, an aromatic polyether dendrimer, or a combination thereof.

9. A pharmaceutical composition comprising the nanoparticle system of claim 4 and a pharmaceutically acceptable excipient.

10. A method of making a nanoparticle system, comprising
contacting multivalent nanoparticle cores comprising multiple reactive end groups with a composition comprising one or more peptides of claim 1 under conditions sufficient to conjugate a plurality of the peptides to the multivalent nanoparticle cores and provide the nanoparticle system.

11. The method of claim 10, wherein the reactive end groups comprise dicyclohexylcarbodiimide, diisopropylcarbodiimide, N-(3-Dimethylaminopropyl)-N′-ethylcarbodiimide, 1,1′-carbonyldiimidazole, N-succinimidyl S-acetylthioacetate, N-succinimidyl-S-acetylthiopropionate, 2-Mercaptoethylamine, sulfosuccinimidyl 4-(N-maleimidomethyl) cyclohexane-1-carboxylate, succinimidyl iodoacetate, succinimidyl 3-(2-pyridyldithio) propionate, N-hydroxysuccinimide ester, N-hydroxy sulfosuccinimide ester, N-y-maleimidobutyryl-oxysulfosuccinimide ester, nitrophenyl ester, tetrafluoro phenyl ester, pentafluorophenyl ester, thiopyridyl ester, thionitrophenyl ester, or a combination thereof.

12. The method of claim 10, wherein the multivalent nanoparticle core comprises a hyperbranched polymer, a dendrimer, a dendron, a hybrid nanoparticle, or a micelle.

13. An immunotherapy method comprising administering to a subject in need thereof the nanoparticle system of claim 1.

14. The immunotherapy method of claim 13, wherein the subject is a human cancer patient or a human patient with an immune disorder.

15. A method of identifying a therapeutic peptide which binds a target receptor, comprising
a) performing phage display with a phage display library to identify candidate target receptor binding peptides,
b) sequencing DNA encoding the candidate target receptor binding peptides,
c) synthesizing the candidate target receptor binding peptides and determining the binding affinity of the synthesized candidate target receptor binding peptides to the target receptor,
d) based on the binding affinities of c), performing computational modeling to determine conserved peptide-receptor binding components, and
e) using the conserved peptide-receptor binding components, performing adaptive-evolution modeling and identifying one or more mutated target receptor binding peptides with optimized peptide-receptor binding components,
f) synthesizing the one or more mutated target receptor binding peptides and determining the binding affinity of the synthesized mutant target receptor binding peptides to the target receptor, and
g) identifying the therapeutic peptide as a peptide from f) having a dissociation constant of 10⁻³ M or lower for the target receptor.

16. The method of claim 15, wherein phage display step a) comprises
i) providing the phage display library expressing a plurality of test peptides,
ii) incubating the phage display library with the target receptor,
iii) selecting phage bound to the target receptor,
iv) amplifying the selected phage of iii), and
v) repeating steps ii) to iv) one or more times to provide the candidate target receptor binding peptides.

17. The method of claim 15, wherein in c) determining the binding affinity to the target receptor comprises biolayer interferometry, or surface plasmon resonance.

18. The method of claim 15, wherein in steps c) and f) synthesizing comprises solid phase synthesis.

19. The method of claim 15, wherein in step d) computational modeling comprises a molecular dynamic simulation, and wherein in step e) adaptive evolution modeling comprises iterative Monte Carlo simulation of the one or more mutated target receptor binding peptides with optimized peptide-receptor binding components.

20. The method of claim 15, further comprising, h) synthesizing the therapeutic peptide and conjugating the therapeutic peptide to a multivalent nanoparticle core.


SEQUENCE LISTING (16 sequences)

SEQ ID NO: 1 - protein, 12 aa, synthetic construct
QVACHWMCTDQT

SEQ ID NO: 2 - protein, 12 aa, synthetic construct
NDAVIKNLLSSN

SEQ ID NO: 3 - protein, 12 aa, synthetic construct
NGSQSDIYRIQT

SEQ ID NO: 4 - protein, 12 aa, synthetic construct
ITLGYDARAPNA

SEQ ID NO: 5 - protein, 12 aa, synthetic construct
VDSYSPPTTSHL

SEQ ID NO: 6 - protein, 12 aa, synthetic construct
HDLGQYIIYGQM

SEQ ID NO: 7 - protein, 12 aa, synthetic construct
DEGHVTSKHYNW

SEQ ID NO: 8 - protein, 12 aa, synthetic construct
AFWKTDGFSRAN

SEQ ID NO: 9 - protein, 15 aa, synthetic construct
YPWRWYYHVTFFCRW

SEQ ID NO: 10 - protein, 12 aa, synthetic construct
TKGHVTSKHYNW

SEQ ID NO: 11 - protein, 13 aa, synthetic construct
RTRWYRGVTMGEW

SEQ ID NO: 12 - protein, 14 aa, synthetic construct
QYFPENRVMERAWR

SEQ ID NO: 13 - protein, 14 aa, synthetic construct
QYFPMTRVMERAWR

SEQ ID NO: 14 - protein, 14 aa, synthetic construct
QYFPENRCMERAWR

SEQ ID NO: 15 - protein, 249 aa, Mus musculus
MHGWLLLVWVQGLIQAAFLATAIGATAGTIDTKRNISAEEGGSVILQCHFSSDTAEVTQVDWKQQDQLLAIYSVDLGWHVASVFSDRVVPGPSLGLTFQSLTMNDTGEYFCTYHTYPGGIYKGRIFLKVQESSDDRNGLAQFQTAPLGGTMAAVLGLICLMVTGVTVLARKDKSIRMHSIESGLGRTEAEPQEWNLRSLSSPGSPVQTQTAPAGPCGEQAEDDYADPQEYFNVLSYRSLESFIAVSKTG

SEQ ID NO: 16 - protein, 244 aa, Homo sapiens
MRWCLLLIWAQGLRQAPLASGMMTGTIETTGNISAEKGGSIILQCHLSSTTAQVTQVNWEQQDQLLAICNADLGWHISPSFKDRVAPGPGLGLTLQSLTVNDTGEYFCIYHTYPDGTYTGRIFLEVLESSVAEHGARFQIPLLGAMAATLVVICTAVIVVVALTRKKKALRIHSVEGDLRRKSAGQEEWSPSAPSPPGSCVQAEAAPAGLCGEQRGEDCAELHDYFNVLSYRSLGNCSFFTETG